(19) United States
(12) Patent Application Publication
(10) Pub. No.: US 2022/0252535 A1
(43) Pub. Date: Aug. 11, 2022

(54) HUMIDITY INITIATED GAS (HIG) SENSORS FOR VOLATILE ORGANIC COMPOUNDS SENSING

(71) Applicant: MASSACHUSETTS INSTITUTE OF TECHNOLOGY, Cambridge, MA (US)

(72) Inventors: Maxwell Robinson, Cambridge, MA (US); Karen K. Gleason, Cambridge, MA (US)

(73) Assignee: MASSACHUSETTS INSTITUTE OF TECHNOLOGY, Cambridge, MA (US)

(21) Appl. No.: 17/592,113

(22) Filed: Feb. 3, 2022

Related U.S. Application Data
(60) Provisional application No. 63/146,249, filed on Feb. 5, 2021.

(51) Int. Cl. G01N 27/22; B01L 3/00

(52) U.S. Cl. CPC G01N 27/226; B01L 3/5023; B01L 3/502715; G01N 33/0047; B01L 2300/0645; B01L 2300/0816; G01N 2027/222; B01L 2300/069

(57) ABSTRACT

Volatile organic compounds (VOCs) can be sensed using humidity-initiated gas (HIG) sensors.

### CLAIM OF PRIORITY

The application claims priority to U.S. Provisional Application No. 63/146,249, filed Feb. 5, 2021, which is incorporated by reference in its entirety.

### FIELD OF THE INVENTION

The invention relates to sensors and methods of detecting an analyte.

### BACKGROUND

Volatile organic compounds (VOCs) are organic chemicals that are present as vapor at room temperature. See, for example, R. Koppman, Volatile Organic Compounds in the Atmosphere (Ed.: Koppman, R.), 1st ed., Blackwell Publication, Oxford, 2007, which is incorporated by reference in its entirety. The VOCs produced within various industries—including petrochemicals, healthcare, food processing, and agriculture—carry information about a source process and so, represent a data stream to inform decision-making. See, for example, H. Zheng, et al., Sci. Total Environ. 2020, 703, 135505; I. A. Hanouneh, et al., Clin. Gastroenterol. Hepatol. 2014, 12, 516; F. Biasioli, et al., TrAC—Trends Anal. Chem. 2011, 30, 968; and A. Cellini, et al., Sensors (Switzerland) 2017, 17, each of which is incorporated by reference in its entirety. Therefore, there is significant academic and commercial interest in improved VOCs sensors technologies.

### SUMMARY

In one aspect, a sensor for detecting an analyte can include a first electrode, a second electrode, and a sensor element including a rough-surfaced material having a coating on the surface of the rough-surfaced material, the coated surface having a hydration surface.

In another aspect, a method of sensing an analyte can include exposing a sensor to an atmosphere having a relative humidity of at least 30%, the sensor including a first electrode, a second electrode, and a sensor element including a rough-surfaced material having a coating on the surface of the rough-surfaced material, the coated surface having a hydration surface, and measuring an electrical property of the sensor to detect the analyte in the atmosphere.

In another aspect, a method of detecting a volatile organic compound can include exposing a sensor to an atmosphere having a relative humidity of at least 30%, the sensor including a first electrode, a second electrode, and a sensor element including a rough-surfaced material having a coating on the surface of the rough-surfaced material, the coated surface having a hydration surface, and measuring an electrical property of the sensor to detect the analyte in the atmosphere includes measuring the impedence of a water layer on or within the hydration surface.

In certain circumstances, the hydration surface can include a surface upon which a thin layer of water forms.

In certain circumstances, the rough-surfaced material can include inorganic particles.

In certain circumstances, the inorganic particles can include silica.

In certain circumstances, the hydration surface can include a plurality of capillaries.

In certain circumstances, the rough-surfaced material can include a capillary-forming material.

In certain circumstances, the capillary-forming material can include a sheet-forming material.

In certain circumstances, the water layer can sorb the analyte.

In certain circumstances, the coating can sorb the analyte.

In certain circumstances, the atmosphere can have a relative humidity of at least 40%, at least 50%, at least 60%, or at least 70%.

In certain circumstances, measuring the electrical property of the sensor to detect the analyte in the atmosphere can include measuring the impedence of a water layer on the hydration surface.

In certain circumstances, measuring the electrical property of the sensor to detect the analyte in the atmosphere can include measuring the impedence of a water layer within the hydration surface.

In certain circumstances, the impedence of the water layer on the hydration surface can change when the water layer sorbs the analyte.

In certain circumstances, the impedence of the water layer in the hydration surface can change when the coating sorbs the analyte.

In certain circumstances, the analyte can be a volatile organic compound, for example, a volatile organic compound that is indicative of a citrus disease.

In other aspects, the sensor and methods described herein can be applied to detect VOCs in a variety of circumstances. For example, VOCs can be detected in food services, healthcare, or environmental monitoring applications.

Other aspects, embodiments, and features will be apparent from the following description, the drawings, and the claims.

### BRIEF DESCRIPTION OF DRAWINGS

FIGS. 1A-1F depict humidity-initiated gas (HIG) sensors to detect volatile organic compounds (VOCs). FIG. 1A shows VOCs detection permits disease diagnosis, including the citrus disease Huanglongbing (HLB) at early stages when it is located at a few leaves (yellow region) out of many thousands. FIG. 1B shows three VOCs have been identified as volatile biomarkers of HLB disease: geranyl acetone (GA, green), linalool (Lin, red), and phenylacetaldehyde (PhA, blue). FIG. 1C shows six requirements of a sensors technology for effective field detection of VOCs, which indicates that no existing technology simultaneously addresses all characteristics well, leading to the development of a sensors concept to meet this need—humidity-initiated gas (HIG) sensors described herein. FIG. 1D shows HIG sensors are prepared on interdigitated electrodes (IDEs) and are composed of a scaffold material, iCVD polymer, and nanoscale water regions at humidity. FIG. 1E shows Type I sensors employ a high surface area inactive scaffold as an ultrathin iCVD polymer growth substrate. At high humidity, an adsorbed water layer forms atop the polymer. Type I sensors use the impedance of this water layer to sense VOCs. FIG. 1F shows Type II sensors employ an iCVD growth scaffold that intercalates water but not VOCs at high humidity. Type II sensors use the impedance of water contained in capillaries to sense VOCs.

FIGS. 2A-2E depict Type I HIG sensors based on fumed silica scaffolds. FIG. 2A shows deposited conformal and ultrathin (<10 nm) polymer on fumed silica (fs) particle films using iCVD to form Type I HIG sensors. The high surface area of the fs permits a larger amount of water to be sorbed as a film at the polymer interface, resulting in a larger impedance signal. FIG. 2B shows a circuit model consisting of a capacitor and series Z_{G} and Z_{B} to transiently fit the impedance spectra of fs coated in 2 nm NP1 when exposed to high relative humidity (RH) of 72%. The circuit model employed demonstrates excellent fits to impedance spectra. FIG. 2C shows employing this circuit model to transiently fit fs/NP1 impedance during exposure to 50 ppm PhA at a constant background RH of 72%. The components of Z_{G} (Q_{G} and n_{G}) measurably respond to HLB VOCs exposure, indicating that both impedance features can be used to sense VOCs. FIG. 2D shows observed long term stability over 14 h of continuous testing for fs/polymer sensors with limited response decay or background drift. FIG. 2E shows fs/NP1 and fs/NP2 with 2 nm polymer thickness demonstrate nearly identical sensing response to GA exposure, indicating that Type I response is highly independent of polymer-VOC affinity as quantified using Flory Huggins interaction parameter (χ).

FIGS. 3A-3C depict a Type I sensor proposed mechanism and response to HLB VOCs. FIG. 3A shows a proposed mechanism where Type I HIG sensor signal derives predominantly from vapor pressure lowering stimulated by VOC sorption with a thin water layer formed at high humidity. Vapor pressure lowering results in water being drawn from the vapor phase to be incorporated within the water layer, resulting in increased water layer thickness. This additional water results in an observable impedance response. FIG. 3B shows a test of fs/NP1 (2 nm) sensor response to each of the three HLB VOCs at constant VOC saturation ratio (SR) of 10%. It was observed that Q_{G} response increases with VOC-water solubility, which is evidence in support of the hypothesis. FIG. 3C shows response in Q_{G}, response in n_{G}, and τ_{G} as features for principle component analysis (PCA). It was observed that PhA can readily be distinguished from GA and Lin, however, GA and Lin cannot be distinguished from one another, which we attribute to their both having similarly low water solubilities. Note that ellipses represent 99% confidence intervals.

FIGS. 4A-4E depict Type II HIG sensors based on a graphene oxide water capillary scaffold. FIG. 4A shows deposit nanoscale iCVD polymer atop graphene oxide (GO) films to form a GO/polymer bilayer. This result can be visualized by a cross-sectional SEM of a GO film with a blanket layer of NP4 on top. Under humid conditions this sensor architecture develops three distinct water regions—an adsorbed water layer, water in polymer, and water intercalated within GO capillaries. FIG. 4B shows a circuit model consisting of a capacitor with series Z_{G} and Z_{B} to accurately fit the impedance spectra of GO coated in 26 nm NP1 at 72% RH. FIG. 4C shows the components of Z_{B} (Q_{B} and n_{B}) measurably respond to HLB VOCs exposure, indicating they can both be used to sense VOCs. FIG. 4D shows long term stability over 14 h of continuous testing for GO/polymer sensors with limited response decay or background drift. FIG. 4E shows when exposed to GA, GO/polymer sensors incorporating a NP2 polymer (χ=0.1) demonstrate 5× the response in Q_{B} and n_{B} as one incorporating NP1 (χ=0.3), suggesting that polymer chemistry is a principal driver of sensor response. Thus, Type II sensor performance can be tuned by polymer selection.

FIGS. 5A-5D depict Type II sensor proposed mechanism and response to HLB VOCs. FIG. 5A shows Type II HIG sensor signal predominantly derives from capillary hydration stimulated by VOC sorption with the contacting polymer layer. VOCs sorb with the polymer layer, displacing water molecules to drive water exit from the polymer layer to within water capillaries. Capillary hydration results in an observable change in impedance, which is the measured Type II response. FIG. 5B shows Type II sensors with non-polar polymer chemistry display increasing detection limits with lower GA-polymer solubility (higher χ). However, Type II sensors including hydrophilic polymers (hydrogen bonding or polar) have low solubility for the HLB VOCs, yet display low detection limits (<20 ppb). One can attribute this behavior to the high water content of hydrophilic polymers relative to the non-polar polymers, which enables hydrophilic polymers a larger Type II response per VOC sorption event. FIG. 5C shows a test of a GO/NP1 (26 nm) sensor response to each of the three HLB VOCs at uniform SR (10%) and background RH (72%). Response in Q_{B} increases with decreasing χ interaction parameter, which supports the hypothesis that polymer-VOC miscibility as a principal driver to Type II response. FIG. 5D shows response in Q_{B}, response in n_{B}, and τ_{B} as features for principle component analysis (PCA). It was observed that the GO/NP1 (Type II) sensor can readily distinguish between each of the three HLB VOCs, in contrast to the fs/NP1 (Type I) sensor (FIG. 3C). Note that ellipses represent 99% confidence intervals.

FIGS. 6A-6C depict HIG sensor performance. Sensors studied fall into sensor categories of bare scaffolds, Type I sensors, and Type II sensors. FIG. 6A shows a key. FIG. 6B shows a log-log plot of detection limits vs response time for GA. It was observed that an iCVD coating on fs to form a Type I sensor slightly improves response time but significantly increases detection limits. Thus, the significant observed stability advantages of Type I sensors (Table 5) are in tension with the higher detection limits offered by a bare fs scaffold. It was also observed that Type I sensor response time is not significantly affected by the polymer coating chemistry. Next, it was observed that an iCVD coating on GO to form a Type II sensor permits a wide range of response times and detection limits based on the iCVD polymer selected. FIG. 6C shows tests performed with each of the three HLB VOCs at 10% SR for a select portion of HIG sensors, and plot PhA:GA selectivity vs Lin:GA selectivity. This plot permits us to visualize sensors that demonstrate GA-selective (green region), Lin-selective (red region), and PhA-selective (blue region) response. Type I sensors, as represented by fs/NP1, demonstrate PhA-selective response. Type II sensors demonstrate Lin- and GA-selective response depending on the polymer selected.

FIG. 7 depicts a sensor as described herein.

FIG. 8 depicts FTIR spectra of hydrogel samples used to determine HEMA composition. The absorbance regions for carbonyl (C═O) and hydroxyl (O—H) stretching are shown on the right and left, respectively. Each sample was dried overnight in a vacuum oven at 60° C. before spectra were taken. Spectra shown are background subtracted, baselined, and normalized to 1. Hydrogel film thicknesses ranged from 500 to 800 nm as measured by ellipsometry.

FIGS. 9A-9C depicts HIG scaffold BET surface area. FIG. 9A shows adsorption experiments with QCM crystals coated in scaffold materials (fs or GO) to measure their specific surface area. FIG. 9B shows butyl acrylate is chosen as the adsorbate molecule for these experiments due to its being the iCVD monomer that comprises NP1. FIG. 9C shows BET plots of a bare QCM crystal, QCM crystal coated in fs, and QCM crystal coated in GO. Bare and GO-coated QCM crystal plots are highly similar, consistent with GO capillaries being inaccessible to butyl acrylate and other iCVD monomers. However, the fs-coated QCM crystal plot is very different from the bare QCM crystal plot, which is the result of the high surface area enhancement provided by fs.

FIG. 10 depicts comparison of NP1 mass deposited on scaffold-coated and bare QCM crystals. NP1 was deposited at identical conditions (see Table 3) on bare, fs-coated, and GO-coated QCM crystals while performing in-situ transient mass measurements. It was observed that mass deposited on a GO-coated crystal is nearly identical to that deposited on a bare crystal, supporting our assertion that iCVD polymer does not deposit within GO capillaries. However, a significant increase in mass deposited on a fs-coated QCM crystal relative to a bare crystal was observed, consistent with polymer growth from inner surfaces of the high surface area fs scaffold film

FIGS. 11A-11D depicts fitting of bare fs and fs/PBA impedance spectra across wide range of RH. FIG. 11A shows an accurate fit of both bare fs and fs/NP1 spectra with a circuit model composed of a capacitor in parallel with series Z_{G} and Z_{B}. Note that this is the same circuit model that is used to fit transient response to VOCs in the main text. FIG. 11B shows measured and fitted spectra for bare fs and fs/NP1 at 72%. Note differences between bare fs and fs/NP1 spectra. This can be observed in higher impedance magnitude, |Z|, and phase shifted to higher frequencies for fs/NP1 relative to bare fs. To explore this further, impedance fits were compiled and analyzed across a wide range of high RH (55-90%). FIG. 11C shows a plot of Q_{G} for bare fs and fs/NP1 across the humidity range. It was observed that Q_{G} is more than 2 orders of magnitude lower for fs/NP1 relative to bare fs. This result can be attributed to a thinner water layer residing on NP1 surfaces of fs/NP1 versus that residing on silica surfaces of bare fs. FIG. 11D shows a plot of n_{G} for bare fs and fs/NP1 across the humidity range. It was observed that n_{G} is nearly double for fs/NP1 relative to bare fs. One can attribute this result to interactions of the water layer with the polymer layer that results in Z_{G} being less resistive. Note that the deposited NP1 layer deposited is 9 nm and that error bars represent standard deviation of at least two sensors studied.

FIGS. 12A-12B depict impedance spectrum and response of interdigitated electrode (IDE) coated in NP1. Note that unlike Type I and Type II sensors, this sensor architecture does not include a scaffold material to sorb additional water from humid environments. FIG. 12A shows an impedance spectrum of an IDE coated in 9 nm NP1 at 72% RH. The impedance magnitude (top) of this scaffold-less sensor is orders of magnitude higher than that of a Type I sensor (FIG. 15) and a Type II sensor (FIGS. 18A-18B) at 72% RH, which is consistent with the scaffold-less sensor sorbing significantly less water. At this low level of water sorption, Z_{B} is not visible and Z_{G} is not fully visible and so, the impedance spectra is dominated by the parallel capacitance. This is demonstrated by the near −90° phase angle across much of the phase angle plot (bottom). Thus, we monitor this capacitance to evaluate a scaffold-less sensor's response. FIG. 12B shows a plot of parallel capacitance during a 4 ppm GA exposure experiment at 72% RH demonstrates a negligible response. Therefore, a scaffold is necessary to sense VOCs using the HIG sensor concept.

FIGS. 13A-13B depict a comparison of Type I sensor (fs/NP1) impedance spectrum and response at 0% and 72% RH. FIG. 13A shows an impedance spectrum of fs/NP1 at 0% RH (gray symbols) and 72% RH (red symbols). Spectra were fit with a circuit model composed of a capacitor in parallel with series Z_{G} and Z_{B} (solid lines). Note that this is the same circuit model that is used to fit transient response to VOCs in the main text for both Type I and Type II sensors. Significantly higher impedance at 0% RH was observed, consistent with water sorbing at sensor surfaces at 72% RH to lower impedance. FIG. 13B shows negligible response at 0% RH, but measurable response at 72% RH. Response at 72% RH is presented as a percent change in Q_{B}. The 0% RH response is presented as percent change in C, since both Z_{G} and Z_{B} are not visible at this RH due to very low accumulation of water at surfaces. Note that the thickness of the NP1 layer is 9 nm.

FIG. 14 depicts testing of bare fs over long time scales. To evaluate the stability and resilience of bare fs VOCs sensors, bare fs to 5 cycles of 2 ppm GA (yellow regions) was exposed over a 14 h testing period at a background RH of 72%. Significantly more background drift and response decay was observed for bare fs relative to a Type I sensor composed of NP2 (FIG. 2D). Thus, a primary advantage of Type I sensors relative to bare fs is significantly improved stability.

FIG. 15 depicts principal component analysis (PCA) features for fs/NP1 classification. Features from fs/NP1 tests with HLB VOCs for principal component analysis (PCA). Features include response in Q_{G}, response in n_{G}, and response time (τ_{G}). All tests were conducted at a saturation ratio (SR) of 10% and background relative humidity (RH) of 72%.

FIGS. 16A-16C depict successful classification by fs/NP1 of HLB VOCs at 20% SR. FIG. 16A shows a response in features of Z_{G} (Q_{G} and n_{G}) that are used for classification. FIG. 16B shows results with 10% SR (FIG. 3B), where a positive relationship between response and VOC water solubility was observed. FIG. 16C shows principal component analysis (PCA) performed on this data, and it was found that complete classification can be achieved at this VOC saturation ratio. Note that ellipses denote 99% confidence intervals. Background RH was 72% for all tests and the thickness of NP1 is 9 nm.

FIGS. 17A-17D depict fitting of bare GO and GO/PBA impedance spectra across wide range of RH. FIG. 17A shows an accurate fit of both bare GO and GO/NP1 spectra with a circuit model composed of a capacitor in parallel with series Z_{G} and Z_{B}. Note that this is the same circuit model that is used to fit transient response to VOCs in the main text for both Type I and Type II sensors. FIG. 17B shows measured and fitted spectra for bare GO and GO/NP1 at 72%. High similarity between bare GO and GO/NP1 impedance spectra VI and phase) at this condition was noted. To explore this further we compiled and analyzed impedance fits across a wide range of high RH (55-90%). Strong similarity was observed between Z_{B} components that include (FIG. 17C) Q_{B} and (FIG. 17D) n_{B}. This similarity can be attributed to two things. First, the blanket layer of NP1 does not act as a significant barrier to water transport from the vapor phase to within GO capillaries. Second, the thin blanket layer of NP1 does not provide its own significant impedance contribution. Note that the deposited NP1 layer deposited is 25 nm and that error bars represent the standard deviation of at least two sensors studied.

FIGS. 18A-18B depict a comparison of Type II sensor (GO/NP1) impedance spectrum and response at 0% and 72% RH. FIG. 18A shows an impedance spectrum of GO/NP1 at 0% RH (gray symbols) and 72% RH (red symbols). Spectra were fit with a circuit model composed of a capacitor in parallel with series Z_{G} and Z_{B} (solid lines). Note that this is the same circuit model that is used to fit transient response to VOCs in the main text for both Type I and Type II sensors. Significantly higher impedance at 0% RH was observed, consistent with water sorbing at sensor surfaces at 72% RH to lower impedance. FIG. 18B shows a negligible response at 0% RH, but measurable response at 72% RH was observed. Response at 72% RH is presented as a percent change in Q_{G}. The 0% RH response is presented as percent change in C, since both Z_{G} and Z_{B} are not well visible at this RH due to very low accumulation of water at surfaces. Note that the thickness of the NP1 layer is 18 nm.

FIG. 19 depicts testing of bare GO over long time scales. To evaluate the stability and resilience of bare GO VOCs sensors, we expose bare GO to 5 cycles of 2 ppm GA (yellow regions) over a 14 h testing period at a background RH of 72%. It was observed that coating GO to form a Type II sensor improves stability, including background drift and response decay (FIG. 4D).

FIG. 20 depicts features from GO/NP1 tests with HLB VOCs for principal component analysis (PCA). Features include response in Q_{B}, response in n_{B}, and response time (τ_{B}). All tests were conducted at a saturation ratio (SR) of 10% and background relative humidity (RH) of 72%.

FIGS. 21A-21B depict an experimental set up for detecting VOCs from solids.

FIGS. 22A-22C depict sensor results for differentiating solid products.

FIG. 23 depicts exemplary VOCs in a solid product used in FIGS. 22A-22C.

FIG. 24 depicts exemplary VOCs in a different solid product used in FIGS. 22A-22C.

FIG. 25 depicts a flow-based sensor.

FIG. 26 depicts a portion of a flow-based sensor.

FIG. 27 depicts a procedure for using a flow-based sensor.

FIG. 28 depicts a sensor system for a solid sample.

FIGS. 29A-29B depict a sensor chamber.

FIG. 30 depicts a list of VOCs in coffee.

FIGS. 31A-31C depict sensor data for coffee samples.

FIGS. 32A-32C depict a summary of coffee sensing results.

### DETAILED DESCRIPTION

A volatile organic compounds (VOCs) sensing concept using humidity-initiated gas (HIG) sensors is described and demonstrated herein. HIG sensors employ the impedance of water assembled at or within sensor surfaces when exposed to high humidity to sense VOCs at low concentration. Examples of two HIG sensor variants are studied here—Type I sensors and Type II sensors. Type I sensors benefit from simplicity, but can be less attractive in terms of key figures of merit (FOMs), including detection limits and response time. Type II sensors are more complex, but are more attractive in terms of key FOMs. Notably, it was observed that best-in-class Type II HIG sensors can achieve <2 min response times and <10 ppb detection limits for geranyl acetone, a VOC linked to the asymptomatic form of Huanglongbing (HLB) citrus disease. Both Type I and Type II sensors benefit from simple assembly from off-the-shelf materials and remarkable stability at high humidity. The HIG sensors can be an attractive alternative to existing VOCs sensors for remote field detection tasks, including, for example, VOCs detection to diagnose HLB citrus disease.

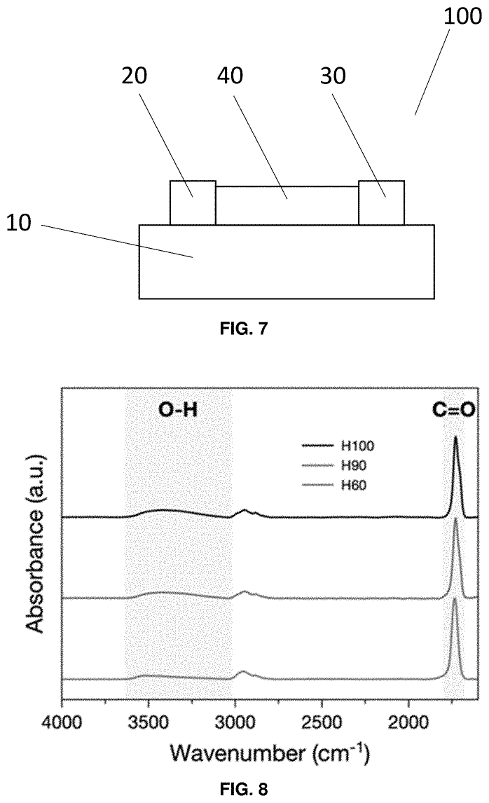

A sensor for detecting an analyte can include a first electrode, a second electrode, and a sensor element including a rough-surfaced material having a coating on the surface of the rough-surfaced material, the coated surface having a hydration surface. The coating on the surface is the coated surface. The coating can include a polymer. Referring to FIG. 7, sensor 100 includes a sensor element 40. Sensor element 40 can be in contact with one or more electrodes 20, 30. The electrodes 20, 30 and sensor element 40 can be arranged on a substrate 10.

Sensor element 40 can include a rough-surfaced material. The rough-surfaced material can be a scaffold for the other components of the device. The rough-surfaced material can have a surface area that is greater than the surface area of the substrate. For example, the rough-surfaced material can have porosity, sheet formations, nanoengineered structures, microengineered structures, capillary structures, or a contoured topology. For example, the rough-surfaced material can be an assembly of particles, an assembly of sheets, or a combination thereof. The assembly of particles can include microspheres, powder, nanoparticles, or nanotubes. The assembly of sheets can be a material that forms sheet layers. Examples of rough-surfaced material can include one or more of inorganic particles, polymer particles, polymer microspheres, metal organic frameworks (MOFs), covalent organic networks (COFs), graphene oxide, clays, or zeolites. In certain circumstances, the rough-surfaced material can include silica particles, alumina particles, polyethylene microspheres, graphene oxide, MOFs, COFs, montmorillonite, or zeolites.

The particles can have a size of less than 10 microns, less than 5 microns, less than 1 micron, less than 100 nm, or less than 50 nm. The particles can have a size of greater than 10 nm, greater than 20 nm, greater than 30 nm, greater than 40 nm, or greater than 50 nm. In certain embodiments, the rough-surfaced material can be hydrophilic. In certain embodiments, the rough-surfaced material can be hydrophobic. The rough-surfaced material can have pores or capillaries. In certain circumstances, the rough-surfaced material can be a capillary-forming material, for example, graphene oxide. The pores or capillaries can have sizes of less than 1 micron, less than 500 nm, less than 250 nm, less than 100 nm, less than 90 nm, less than 80 nm, less than 70 nm, less than 60 nm, or less than 50 nm.

The rough-surfaced material can be a scaffold for a coating. The rough-surfaced material can have a coated surface. The coating of the coated surface can be a polymer, a zeolite, a grafted polymer, an ionic liquid, a covalent organic network, a self-assembled monolayers (SAMs), or material formed by atomic layer deposition (ALD), or molecular layer deposition (MLD). The coating can be on a portion of the surface of the rough-surfaced material. In certain embodiments, the coating can be on a majority of the surface of the rough-surfaced material.

The coating can be a coating having a thickness of less than 1 micron, less than 500 nm, less than 250 nm, less than 100 nm, less than 50 nm, or less than 30 nm. The coating having a thickness of greater than 1 nm, greater than 5 nm, greater than 10 nm, greater than 15 nm, greater than 20 nm, greater than 25 nm, or greater than 30 nm. The coating can be a vapor deposited polymer, for example, as described in U.S. Pat. No. 9,448,219, which is incorporated by reference in its entirety.

In certain circumstances, the polymer can be a polymer or co-polymer including one or more of the monomers selected from the group consisting of maleic anhydride, N-vinyl-2-pyrrolidone, p-bromophenyl methacrylate, pentabromophenyl methacrylate, N-vinyl carbazole, p-divinyl benzene, styrene, alpha methyl styrene, 2-chlorostyrene, 3-chlorostyrene, 4-chlorostyrene, 2,3-dichlorostyrene, 2,4-di chlorostyrene, 2,5-dichlorostyrene, 2,6-dichlorostyrene, 3,4-dichlorostyrene, 3,5-dichlorostyrene, 2-bromostyrene, 3-bromostyrene, 4-bromostyrene, 2,3-dibromostyrene, 2,4-dibromostyrene, 2,5-dibromostyrene, 2,6-dibromostyrene, 3,4-dibromostyrene, 3,5-dibromostyrene, methyl acrylate, n-butyl acrylate, n-pentyl acrylate, n-hexyl acrylate, n-heptyl acrylate, n-octyl acrylate, 2-ethylhexyl acrylate, perfluorocyclohexylmethyl acrylate, benzyl acrylate, 2-hydroxyethyl acrylate, dimethylaminoethyl acrylate, Et3DMAA (N,N-dimethylacetoacetamide), sec-butyl acrylate, tert-butyl acrylate, isobornyl acrylate, ethylene glycol diacrylate, methyl methacrylate, ethyl methacrylate, n-propyl methacrylate, n-butyl methacrylate, isobutyl methacrylate, n-pentyl methacrylate, n-hexyl methacrylate, n-heptyl methacrylate, sec-butyl methacrylate, tert-amyl methacrylate, t-butyl methacrylate, dimethylaminoethyl methacrylate, hydroxyethyl methacrylate, cyclohexyl methacrylate, benzyl methacrylate, isobornyl methacrylate, glycidyl methacrylate, ethylene glycol dimethacrylate, methacrylic acid, styrene, alpha-methyl styrene, ortho-methyl styrene, meta-methyl styrene, para-methyl styrene, para-ethyl styrene, 2,4-dimethyl styrene, 2,5-dimethyl styrene, m-divinylbenzene, p-divinylbenzene, vinylimidazole, 1,4-divinyloxybutane, diethylene glygol divinyl ether, 1,5-hexadiene-3,4-diol, methyl trans-cinnamate, N-morpholinoethyl acrylate, 2-morpholinoethyl methacrylate, 2-isocyanatoethyl methacrylate, 2-sulfoethyl methacrylate, 2-methoxyethyl methacrylate, 2-(tert-butylamino)ethyl methacrylate, 2-ethoxyethyl methacrylate, 2-chloroethyl methacrylate, 2-hydroxypropyl methacrylate, 2-diethylaminoethyl methacrylate, cyclopentyl methacrylate, 2-(diisopropylamino)ethyl methacrylate, 2-bromoethyl methacrylate, 2-phenylethyl methacrylate and 4-vinylpyridine.

In certain circumstances, the polymer can be a copolymer. The copolymer can be a random copolymer or a block copolymer. The block copolymer can be a diblock copolymer, a triblock copolymer, or a tetrablock copolymer.

In certain circumstances, the polymer can include a crosslinker.

In certain circumstances, the polymer can include a crosslinker selected from the group consisting of di(ethylene glycol) di(vinyl ether), ethyleneglycol diacrylate, ethyleneglycol dimethacrylate, di-, tri- or tetraethylen-glycol diacrylate, di-, tri- or tetraethylen-glycol dimethacrylate, allyl acrylate, allyl methacrylate, a C2-C8-alkylene diacrylate, C2-C8-alkylene dimethacrylate, divinyl ether, divinyl sulfone, di- and trivinylbenzene, trimethylolpropane triacrylate or trimethacrylate, pentaerythritol tetraacrylate or tetramethacrylate, bisphenol diacrylate or dimethacrylate, methylene bisacrylamide, methylene bismethacrylamide, ethylene bisacrylamide, ethylene bismethacrylamide, triallyl phthalate, and diallyl phthalate.

The electrodes can be interdigitated electrodes. The spacing between the electrodes can be about 0.25 mm, 0.5 mm, 0.75 mm, 1.0 mm, 1.25 mm, 1.5 mm, 1.75 mm, 2.0 mm, or 5.0 mm. The spacing between the digits of an electrode can be about 1.0 mm, 2.0 mm, 3.0 mm, 4.0 mm, or 5.0 mm. The electrodes can be any conductive material, for example, a metal, semiconductor, or conductive polymer. The electrodes can be stainless steel, gold, platinum, or palladium.

The substrate can include silicon, silicon nitride, silicon oxide, glass, sapphire, polystyrene, polyimide, epoxy, polynorbornene, polycyclobutene, polymethyl methacrylate, polycarbonate, polyvinylidene fluoride, polytetrafluoroethylene, polyphenylene ether, polyethylene terephthalate, polyethylene naphthalate, polypyrrole, or polythiophene.

The coated surface includes a hydration surface. The hydration surface is a surface of the structure that will accumulate a film of water on the surface from water vapor in the surrounding environment. The rough-surfaced material or coating, or both, having a hydration surface can form the film of water when exposed to an atmosphere having a relative humidity (RH) of at least 20%, at least 30%, at least 40%, at least 50%, at least 60%, at least 70%, at least 80%, or at least 90%. The hydration surface can include water contained in the coating. For example, the film of water can interpenetrate the hydration surface to intercalate in the rough-surfaced material. In another example, the film of water can form on the surface of the coating on the rough-surfaced material. The formation of the film of water results in the active sensor. The film of water can be continuous over the surface, can pass through pores or structures in the surface, or combinations thereof. The film of water can be a thin film of water. For example, the film of water can have a thickness of less than 100 nm, less than 90 nm, less than 80 nm, less than 70 nm, less than 60 nm, less than 50 nm, less than 40 nm, or less than 30 nm. In certain circumstances, the hydration surface can include a surface upon which a thin layer of water forms.

The sensor can operate at room temperature or ambient temperature, or at an elevated temperature, for example, elevated relative to room temperature or ambient temperature. For example, the temperature can be less than 80 degrees C., less than 75 degrees C., less than 70 degrees C., less than 65 degrees C., less than 60 degrees C., less than 55 degrees C., less than 50 degrees C., less than 45 degrees C., less than 40 degrees C., less than 35 degrees C., or less than 30 degrees C.

The analyte can be in an atmosphere that is exposed to the sensor. In certain circumstances, the analyte can be a component of a sample in a gas carrier. The gas carrier can be air. The gas carrier including a sample for analysis can pass over the surface of the sensor in a closed environment isolated from other interfering sources. The sample can be pulsed over the sensor. The pulses can have a duration of less than 10 minutes, less than 9 minutes, less than 8 minutes, less than 7 minutes, less than 6 minutes, less than 5 minutes, less than 4 minutes, less than 3 minutes, less than 2 minutes, or less than 1 minute. The sensor can be purged by exposure to a gas carrier that does not include the sample.

The sensor evidences a response to an analyte, for example, a volatile organic compound, through changes in impedance of the water in the film. In certain circumstances, the water layer, the coating, or both can sorb the analyte, directly altering the impedance. The sorbtion can be adsorption or absorption. In certain circumstances, the coating can sorb the analyte. Sorbtion of the analyte can include dissolving the analyte in the water layer or the coating. The sorbtion of the analyte alters the impedence of the water. Examples of impedance measurement are described in the examples below. Impedance spectra of water can be measured over a frequency range of 100 mHz to 10 kHz. The impedance spectra change upon exposure of the sensor to analytes, such as VOCs. The impedance changes can be analyzed, for example, by principle component analysis, to identify particular analytes.

As a consequence of the sorption, the method can include measuring the electrical property of the sensor to detect the analyte in the atmosphere can include measuring the impedence of a water layer on the hydration surface or measuring the impedence of a water layer within the hydration surface.

In another aspect, a method of sensing an analyte can include exposing a sensor to an atmosphere having a relative humidity of at least 30%, the sensor including a first electrode, a second electrode, and a sensor element including a rough-surfaced material having a polymer coating on the surface of the rough-surfaced material, the polymer-coated surface having a hydration surface, and measuring an electrical property of the sensor to detect the analyte in the atmosphere.

In another aspect, a method of detecting a volatile organic compound can include exposing a sensor to an atmosphere having a relative humidity of at least 30%, the sensor including a first electrode, a second electrode, and a sensor element including a rough-surfaced material having a polymer coating on the surface of the rough-surfaced material, the polymer-coated surface having a hydration surface, and measuring an electrical property of the sensor to detect the analyte in the atmosphere includes measuring the impedence of a water layer on or within the hydration surface.

In certain circumstances, the analyte can be a volatile organic compound, for example, a volatile organic compound that is indicative of a citrus disease. In other aspects, the sensor and methods described herein can be applied to detect VOCs in a variety of circumstances. For example, VOCs can be detected in food services, healthcare, or environmental monitoring applications.

One demonstrative application of VOCs sensing in agriculture is the detection of the citrus disease Huanglongbing (HLB) to stop its spread. See, for example, T. Gottwald, et al., Proc. Natl. Acad. Sci. U.S.A 2020, 117, 3492; and A. A. Aksenov, A. Pasamontes, D. J. Peirano, W. Zhao, A. M. Dandekar, O. Fiehn, R. Ehsani, C. E. Davis, Anal. Chem. 2014, 86, 2481, each of which is incorporated by reference in its entirety. Notably, HLB's spread within Florida has resulted in an epidemic that has reduced state-wide citrus plantings from 750,000 acres in 2000 to 476,000 acres in 2014. See, for example, A. W. Hodges, T. H. Spreen, IFAS Ext. 2012, 1, which is incorporated by reference in its entirety. HLB now threatens to reach epidemic status in other high-volume citrus producing regions, including California where 85% of U.S. fresh citrus is produced. HLB has a lengthy asymptomatic stage that can last up to 4 years and so, early detection is critical to stemming its spread. Recent economic models indicate that early HLB detection followed by swift removal and replacement of HLB-positive trees enables profitable citrus grove operation. See, for example, T. Gottwald, et al., Proc. Natl. Acad. Sci. U.S.A. 2020, 117, 3492, which is incorporated by reference in its entirety. However, the bacteria that causes HLB is often present in only a few leaves of tens of thousands in the asymptomatic stage and so, the currently employed leaf-based polymerase chain reaction (PCR) diagnostic fails to adequately diagnose HLB at early stages.

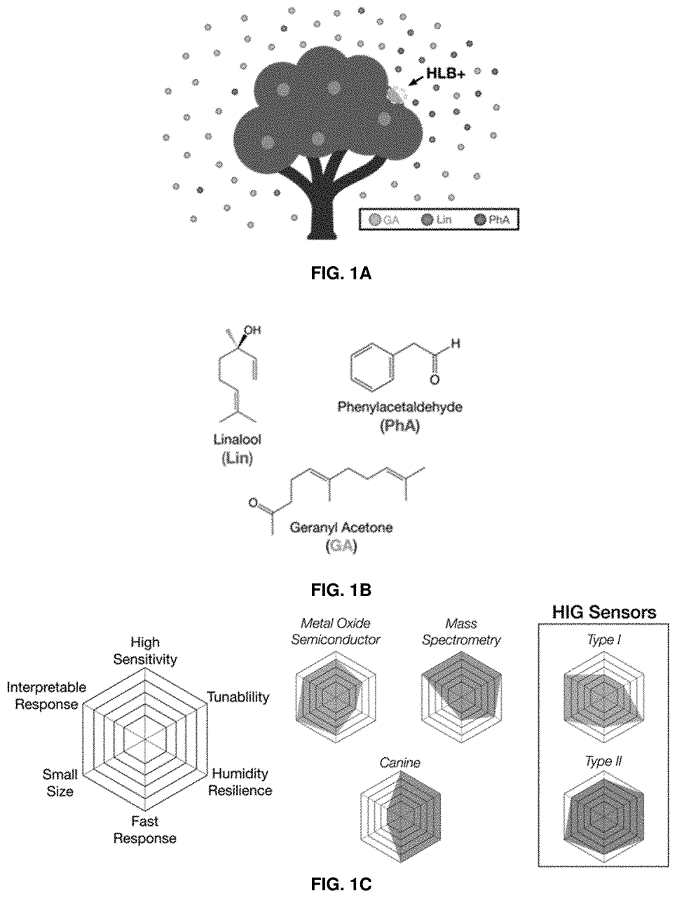

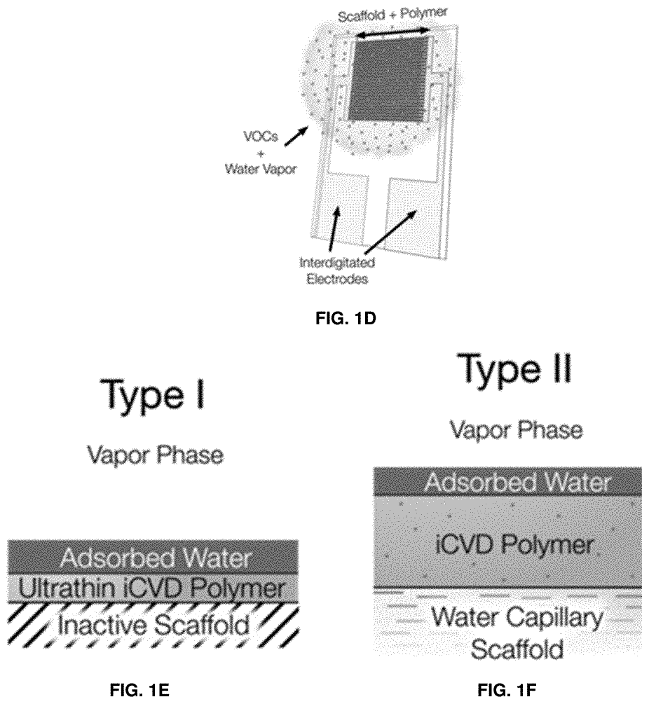

VOCs detection represents a promising avenue for early HLB diagnosis. The concentrations of VOCs released by a citrus tree are modified during healthy as well as during asymptomatic, mild, and severe HLB stages. See, for example, A. A. Aksenov, et al., Anal. Chem. 2014, 86, 2481, which is incorporated by reference in its entirety. These volatiles are available as a detectable cloud surrounding an affected citrus tree that permit HLB diagnosis at the scale of whole plants, in contrast to PCR (FIG. 1A). Mass spectrometry studies have identified VOCs strongly associated with asymptomatic HLB onset, which include geranyl acetone (GA, downregulated), linalool (Lin, upregulated), and phenylacetaldehyde (PhA, upregulated) (FIG. 1B).

To date, asymptomatic HLB has been diagnosed by VOCs detection in a citrus grove environment using mass spectrometry and canine detection. See, for example, T. Gottwald, et al., Proc. Natl. Acad. Sci. U.S.A 2020, 117, 3492; and A. A. Aksenov, et al., Anal. Chem. 2014, 86, 2481, each of which is incorporated by reference in its entirety. However, applications such as HLB detection place multiple requirements on a VOCs sensors technology beyond simply the ability to detect the asymptomatic stage. As described herein, six key requirements for sensors used to perform remote field detection, including high sensitivity, interpretable response, tunability, small size, fast response, and humidity resilience (FIG. 1C and Table 2). Current VOCs sensors do not meet all of these requirements simultaneously. Mass spectrometry is highly sensitive, but can require prohibitively large and/or centralized equipment as well as lengthy setup, test, and data analysis times. See, for example, A. A. Aksenov, et al., Anal. Chem. 2014, 86, 2481, which is incorporated by reference in its entirety. Like mass spectrometry, canine detection is highly sensitive, however canines are large and must be transported on-site to perform each detection task. More importantly, the specific mechanisms that underpin canine detection of VOCs are not well understood. Interpretability raises confidence in performance, since high interpretability implies low risk of poor performance arising from previously unknown contributing factors. See, for example, X. Jia, L. Ren, J. Cai, Med. Phys. 2020, 47, 1, which is incorporated by reference in its entirety. Thus, low response interpretability may result in low technology adoption for applications in which resulting decisions carry significant cost, such as the decision to remove a revenue-generating citrus tree.

On the other hand, smaller-sized VOCs sensors technologies, including oxide semiconductor (MOS) sensors, are typically designed to detect a precise molecule or class of molecules. See, for example, J. W. Yoon, J. H. Lee, Lab Chip 2017, 17, 3537, which is incorporated by reference in its entirety. The significant time and cost to customize to a particular application decreases tunability. More importantly MOS sensors have low humidity resilience and so, are not well-suited to detection tasks in uncontrolled outdoor field environments that are often at high humidity. Herein, a new gas sensing concept that we believe addresses all requirements for remote detection of VOCs and so, may provide for many applications including HLB diagnosis is described.

At high humidity, water vapor accumulates at materials interfaces to form nanoscale, water-rich regions. Such regions display characteristic impedance features. See, for example, Z. Wang, et al., Nanotechnology 2011, 22, which is incorporated by reference in its entirety. Herein, we incorporate nanoscale water-rich regions—including water sorbed as films and intercalated within capillaries—into new a VOCs sensors class that we call humidity-initiated gas (HIG) sensors. The HIG sensors presented here can be assembled atop an interdigitated electrode (IDE) and can be composed of a scaffold coated with a nanoscale polymer film (2-30 nm) (FIG. 1D). We employ initiated chemical vapor deposition (iCVD) to coat scaffolds with polymer. See, for example, H. Matsumura, et al., In Catalytic Chemical Vapor Deposition, Wiley-VCH Verlag GmbH & Co. KGaA, 2019, pp. 179-247, which is incorporated by reference in its entirety. When exposed to humidity, water-rich regions are formed with the scaffold and polymer, resulting in observable impedance features. These water impedance features are modified by VOCs sorption with HIG sensor volumes and interfaces, resulting in a measurable response to VOCs. Notably, unlike current VOCs sensing technologies that treat water as an undesirable contaminant, HIG sensors exploit ambient water vapor to sense VOCs at low concentrations. Moreover, since HIG sensors do not employ electron conduction to detect VOCs, they are distinct from existing small-sized sensors that require sophisticated conducting or semi-conducting materials.

Two types of HIG sensors are explored—Type I and Type II. Type I sensors employ a high surface area inactive scaffold coated with an ultrathin (<10 nm) polymer (FIG. 1E). Addition of the polymer layer significantly improves sensor stability. Type I sensor impedance used to sense VOCs derives from a water layer adsorbed atop the polymer-coated scaffold at high humidity. Accordingly, Type I sensor response increases with degree of water-VOC solubility.

Type II sensors employ an iCVD polymer growth scaffold that can contain small capillaries that intercalate water but not VOCs (FIG. 1F). Type II sensor impedance features used to sense VOCs derive from water intercalated within capillaries at high humidity. It was found that Type II response significantly increases with increasing polymer-VOC solubility and so, Type II selectivity can be significantly tuned through iCVD polymer selection. The ability of HIG sensors to detect and distinguish the HLB-associated VOCs was tested, and it was found that HIG sensors—particularly Type II sensors—are an attractive architecture for use in remote field detection tasks, including HLB diagnosis, diagnosis of other plant diseases, and other high impact applications.

We explore the effects of nanoscale iCVD polymers deposited on scaffolds used to make Type I and Type II HIG sensors (Table 1). We select three classes of well-studied iCVD polymers: polar, non-polar, and hydrogen bonding. For example, H. Matsumura, et al., In Catalytic Chemical Vapor Deposition, Wiley-VCH Verlag GmbH & Co. KGaA, 2019, pp. 179-247, which is incorporated by reference in its entirety. Setpoints used to deposit each iCVD polymer studied are presented in table form (Table 3). For the polar surface modification, P1, the homopolymer of the monomer, cyanoethyl acrylate, was used. For the four non-polar surface modifications, NP1 to NP4, homopolymers of the monomers butyl acrylate, cyclohexyl methacrylate, benzyl methacrylate, and 1,3,5,7-tetravinyl-1,3,5,7-tetramethylcyclotetrasiloxane, respectively, were used. The hydrogen bonding polymers are hydrogels formed from the iCVD copolymerization of hydroxyethyl methacrylate (HEMA) with the crosslinking monomer ethylene glycol diacrylate (EGDA), where H100 represents a homopolymer of HEMA (100 mol % HEMA) and H0 represents a homopolymer of EGDA (0 mol % EGDA). HEMA composition in hydrogels was determined using Fourier transform infrared (FTIR) measurements as has been described previously (FIG. S1). See, for example, K. Chan, K. K. Gleason, Langmuir 2005, 21, 8930; J. L. Yagüe, K. K. Gleason, Soft Matter 2012, 8, 2890; and W. Li, et al., ACS Appl. Mater. Interfaces 2019, 11, 5668, each of which is incorporated by reference in its entirety.

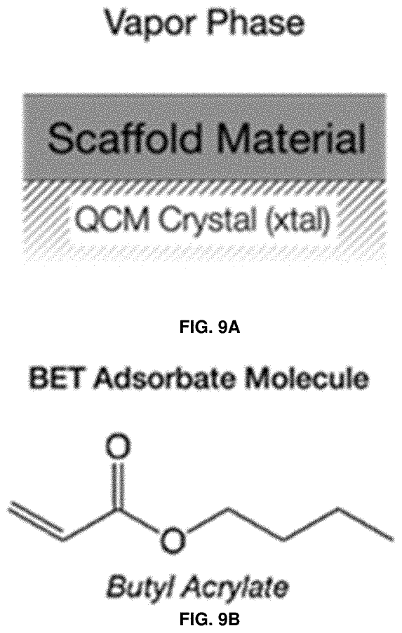

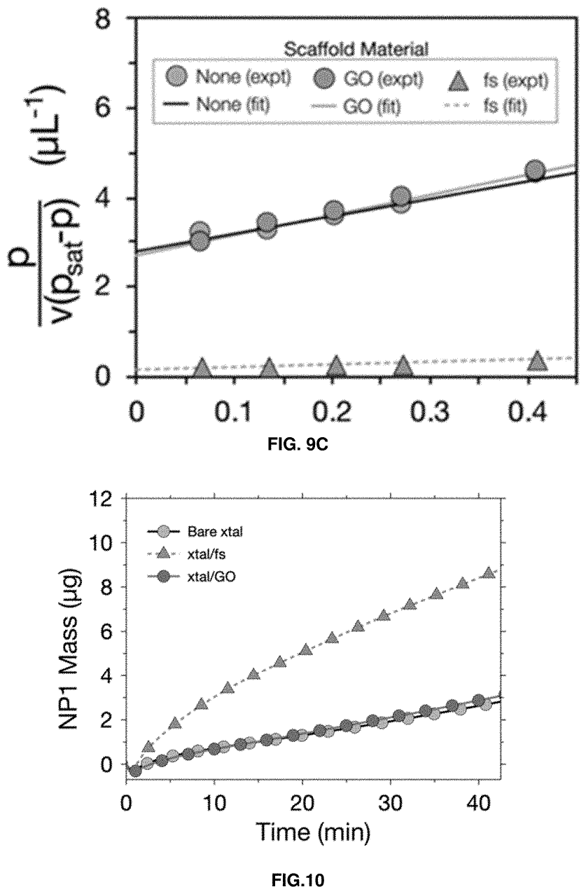

To enable VOCs sensing, Type I HIG sensors require a scaffold to adsorb water vapor. A high surface area scaffold material was employed to greatly increase the total amount of adsorbed water and resulting sensing signal. Herein, fumed silica (fs) nanoparticle films are used as Type I sensor scaffolds due to fs being electrically insulating and having high specific surface area. Using vacuum adsorption measurements and Brunauer-Emmett-Teller (BET) theory (FIGS. 9A-9C), a specific surface area of 170 m²/g was measured for all fs films used in studies herein (Table 4).

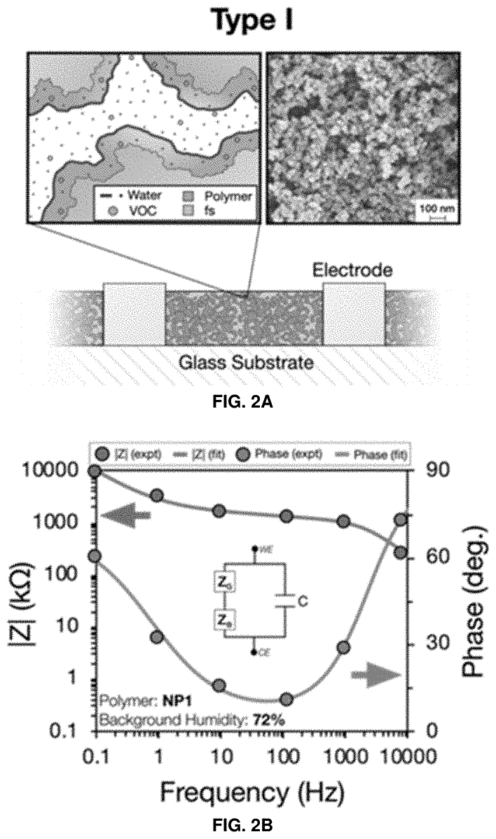

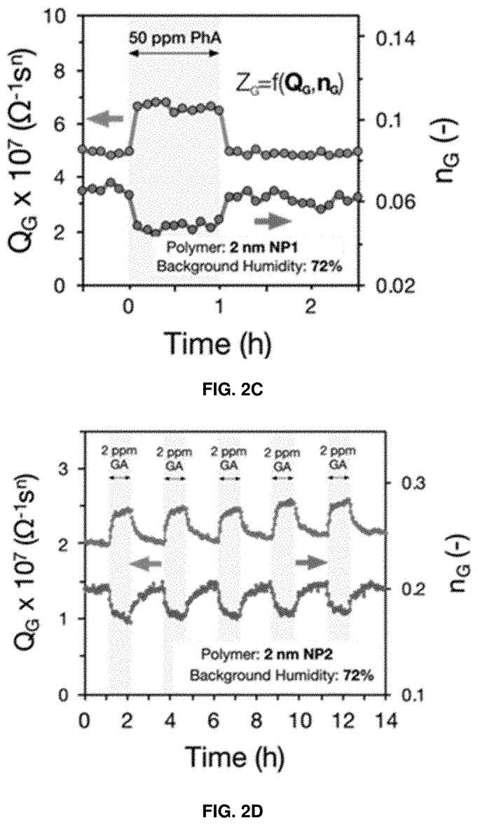

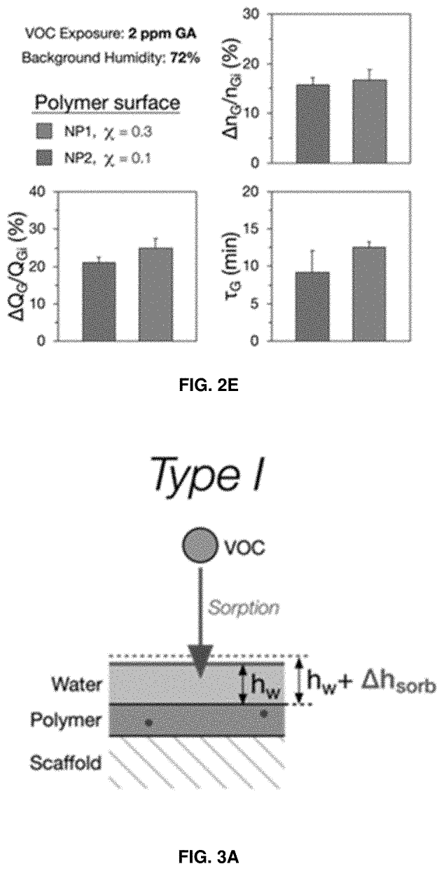

The morphology of an ultrathin layer of iCVD polymer deposited on fs scaffolds was explored. First, in-situ quartz crystal microbalance (QCM) mass measurements were employed to demonstrate that the mass of iCVD-deposited NP1 is >3× larger for fs-coated crystal relative to a bare crystal (FIG. 10). This observation is consistent with the high surface area fs film providing increased sites for iCVD polymer deposition. Second, we drop cast fs on an interdigitated electrode (IDE) and subsequently deposit an ultrathin (˜5 nm) coating of NP1 to create a modified fs scaffold surface. This sample was then imaged using scanning electron microscopy (SEM, Zeiss Sigma 300 VP) to demonstrate that iCVD polymer-modified fs remains nanoporous (FIG. 2A). Thus, ultrathin iCVD polymer coats the inner surfaces of a fs scaffold. The result is a high surface area material to adsorb water vapor but that now also exhibits the surface chemistry of the selected iCVD polymer. IDEs coated in fs and then subsequently coated in ultrathin iCVD polymer are referred to herein as Type I sensors.

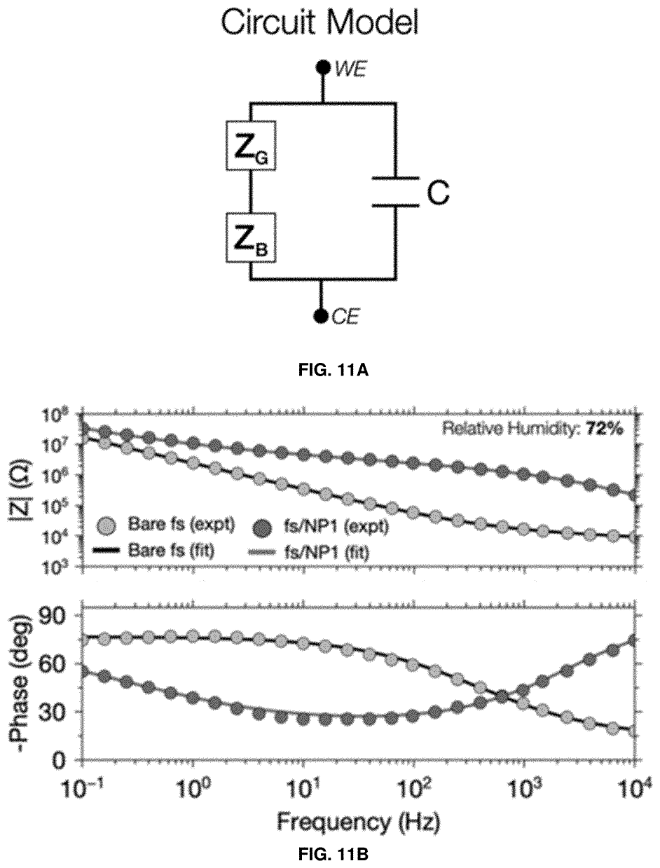

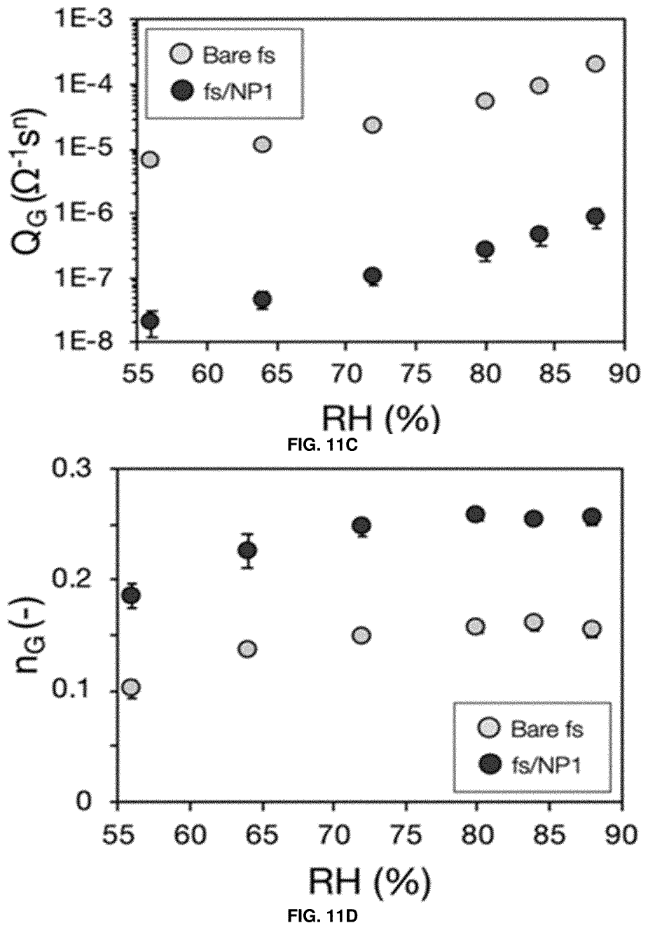

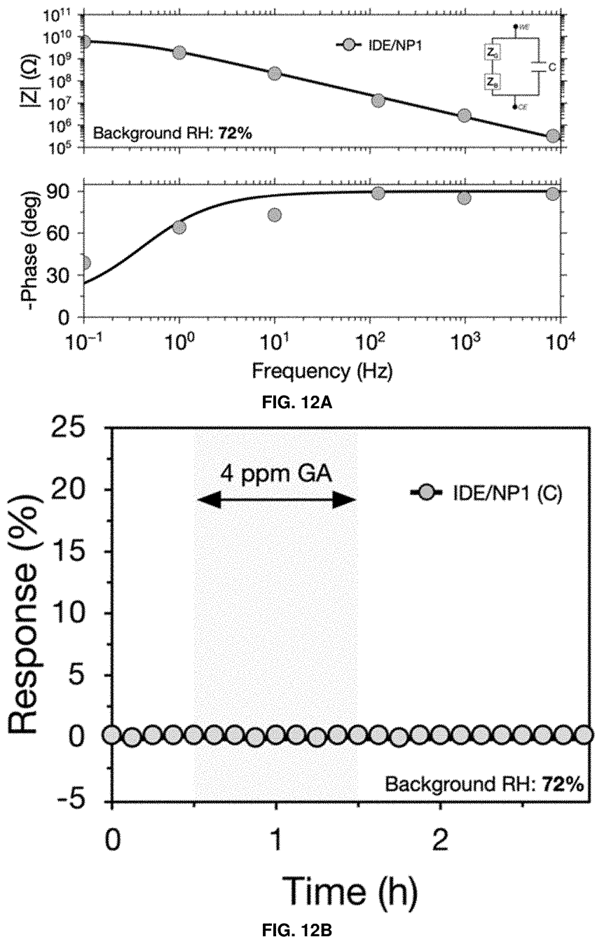

Next, we model the impedance spectra of bare fs and Type I sensors in humid conditions. When exposed to high relative humidity (RH) below saturation, surfaces adsorb a nanoscale multilayer water film displaying two signature impedance features, corresponding to proton conduction via the Grotthus transfer reaction (GTR), Z_{G}, and the bulk-like impedance behavior of water, Z_{B}. See, for example, H. Bi, et al., Sci. Rep. 2013, 3, 1, which is incorporated by reference in its entirety. At higher frequencies, Z_{G} is observed. At lower frequencies, Z_{B} is observed. Z_{G} and Z_{B} are well approximated by separate constant phase elements, defined by

$${Z}_{j} = \frac{1}{{{Q}_{j} ( ω i )}^{{n}_{j}}} \qquad ( 1 )$$

where ω is the angular frequency (rad/s), Qⱼ is the admittance (1/Zⱼ) at 1 rad/s, and nⱼ is the ideality constant with value between 0 and 1 to represent purely resistive and capacitive phenomena, respectively. j is either G or B to denote Z_{G} or Z_{B}, respectively. As has been done with other high surface area materials at humidity, bare fs and Type I impedance spectra were fit with a circuit model composed of a capacitor in parallel with series Z_{G} and Z_{B}. See, for example, Y.-C. Yeh, et al., Commun. Am. Ceram. Soc. 1989, 72, 1472, which is incorporated by reference in its entirety. The capacitor represents contributions from the measuring instrument and the vapor phase. This model achieves excellent fits from 100 mHz to 10 kHz across a wide range of high RH (55-90%) for both bare fs and a Type I sensor composed of NP1 (FIGS. 11A-11D). Note that water impedances, Z_{G} and Z_{B}, are not well visible for a scaffold-free IDE coated in iCVD polymer at 72% RH (FIGS. 12A-12B). Thus, the inclusion of a scaffold material to sorb additional water is necessary to enable the appearance of water impedances. 72% RH was selected for all subsequent VOCs sensing, since at this condition Type I sensor peak phase of Z_{G} is centered within the 100 mHz-10 kHz test frequency range (FIG. 2B).

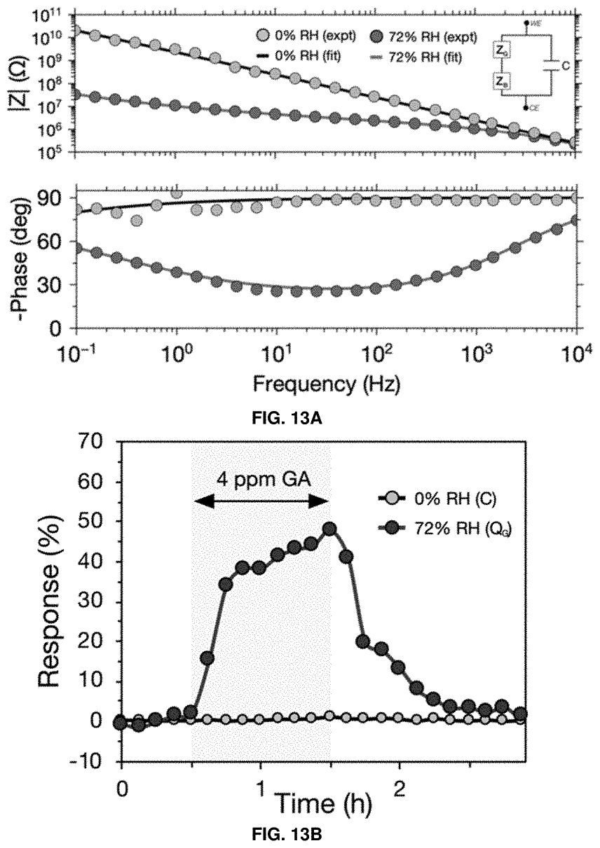

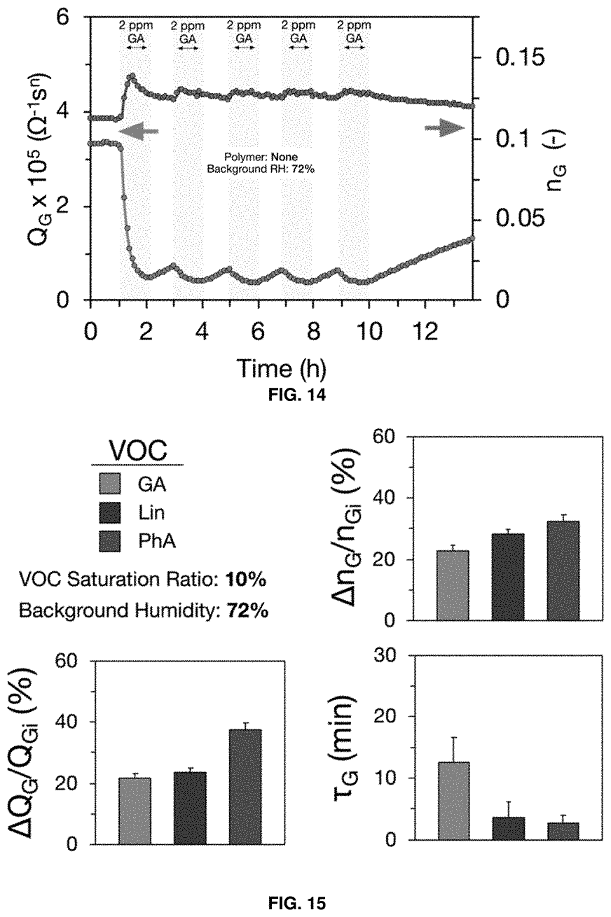

Next, a Type I sensor composed of 2 nm NP1 (fs/NP1) was prepared and its VOCs sensing capabilities were explored. To do this, the sensor's impedance spectra were measured at regular time intervals and fit to derive values of Q_{G} and n_{G} during a 50 ppm PhA exposure experiment at constant background RH of 72% (FIG. 2C). During PhA exposure, both Q_{G} and n_{G} changed in response, were response is defined as percent change in value. Thus, Type I sensors can be used to detect VOCs. Moreover, a sensor that comprises an iCVD polymer but no scaffold material does not produce a measurable response to VOCs and thus, a scaffold material is required to observe a response to VOCs (FIGS. 12A-12B). Furthermore, it was observed that a Type I sensor yields negligible response to VOCs at 0% RH but does yield response at 72% RH (FIGS. 13A-13B). Therefore, Type I sensor response is initiated by ambient water vapor and so, Type I sensors can be classified as HIG sensors.

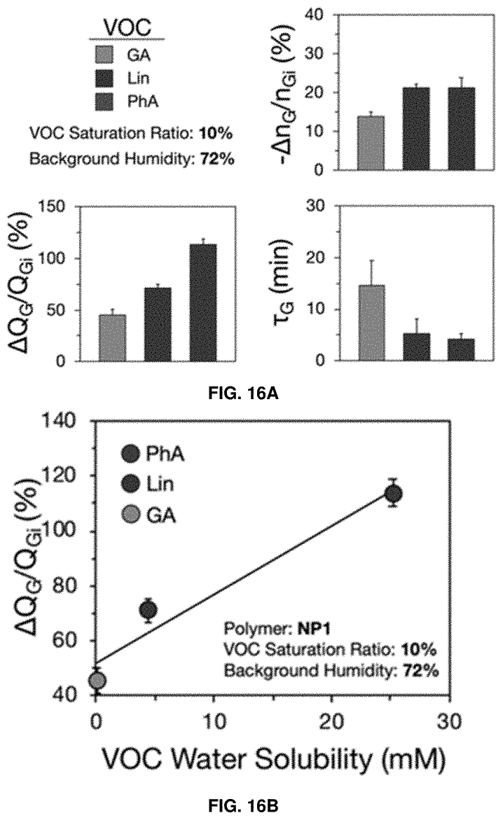

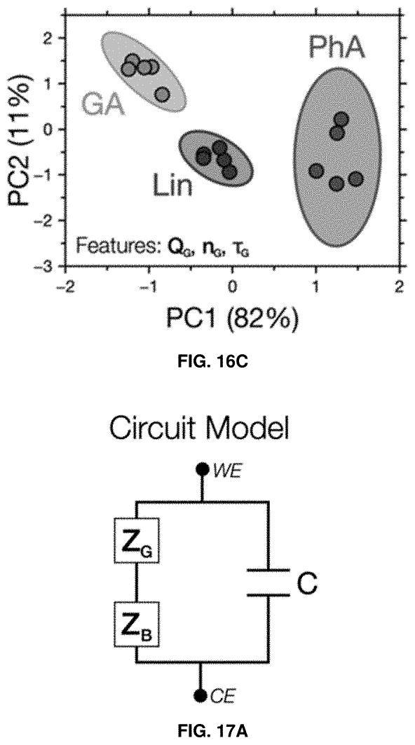

Type I sensor stability was then assessed by testing over long time scales. Stability can be defined in two ways: response decay and background drift. Response decay is the percentage decrease in response over the test period. Background drift is the absolute percentage change in background signal over the test period. fs coated with 2 nm NP2 (fs/NP2) over a 14 h test period that includes five 1 h exposures to 2 ppm GA at 72% background RH were tested (FIG. 2D). From this test, limited response decay in Q_{G} (5%) and more significant response decay in n_{G}(20%) was observed. Minor background drift in Q_{G} (7%) and n_{G} (1%) was also observed. Furthermore, a similar test over long time scales with bare fs (FIGS. 16A-16C) was performed and observe that fs/NP2 demonstrates significantly improved stability (Table 5). For example, fs/NP2 displays 80% less response decay and 90% less background drift in Q_{G} relative to bare fs. Thus, a primary advantage of Type I sensors versus bare fs is greatly improved stability.

To test the effects of polymer chemistry on Type I sensor performance, responses of fs coated in NP1 (fs/NP1) and NP2 (fs/NP2) were compared, which have different miscibility for GA. GA-polymer miscibility can be estimated using the Flory Huggins interaction parameter, χ, defined as

$$χ = \frac{\tilde{V} {A}_{VP}}{RT} \qquad ( 2 )$$

where v is the molar volume of the VOC solute, R is the gas constant (8.3145 J mol⁻¹K⁻¹), and T is the absolute temperature (K). A_{VP} is the Hansen solubility criteria for the VOC-polymer combination, defined as (see, C. M. Hansen, In Hansen Solubility Parameters: A Users Handbook, Second Edition, CRC Press, Boca Raton, 2007, pp. 27-43, which is incorporated by reference in its entirety).

A_{VP}=(δ_{d,V}−δ_{d,P})²+0.25(δ_{p,V}−δ_{p,P})²+0.25(δ_{h,V}−δ_{h,P})² (3)

where δ_{d,i}, δ_{p,i}, and δ_{h,i} are the dispersive, polar, and hydrogen bonding Hansen solubility parameters. Hansen solubility parameters were determined using the Hoy method. See, for example, D. W. Van Krevelen, K. te Nijenhhuis, In Properties of Polymers, Elsevier, 2009, pp. 189-225, which is incorporated by reference in its entirety. χ for GA-NP1 and GA-NP2 was estimated to be 0.29 and 0.11, respectively. Both polymer-GA combinations have χ<0.5, a criteria for solubility. See, for example, D. W. Van Krevelen, K. te Nijenhhuis, In Properties of Polymers, Elsevier, 2009, pp. 189-225, which is incorporated by reference in its entirety. So, GA is expected to solubilize within both polymers. However, χ of NP1-GA is nearly 3× that of NP2-GA and so, NP2 is expected to solubilize more GA than NP1. Therefore, if Type I response derives largely from degree of VOC-polymer sorption, then we would expect the responses of fs/NP1 and fs/NP2 to be significantly different. To evaluate, we test fs/NP1 and fs/NP2 with 2 ppm GA at 72% background RH, and then compare responses and response times (FIG. 2E). Response in both Q_{G} and n_{G} are observed to be statistically identical for fs/NP1 and fs/NP2. Further, response time is defined as τ_{G}, the 1/e time constant for Q_{G}, and τ_{G} was observed to also be statistically identical for fs/NP1 and fs/NP2. Thus, polymer-VOC miscibility does not significantly affect Type I sensor performance.

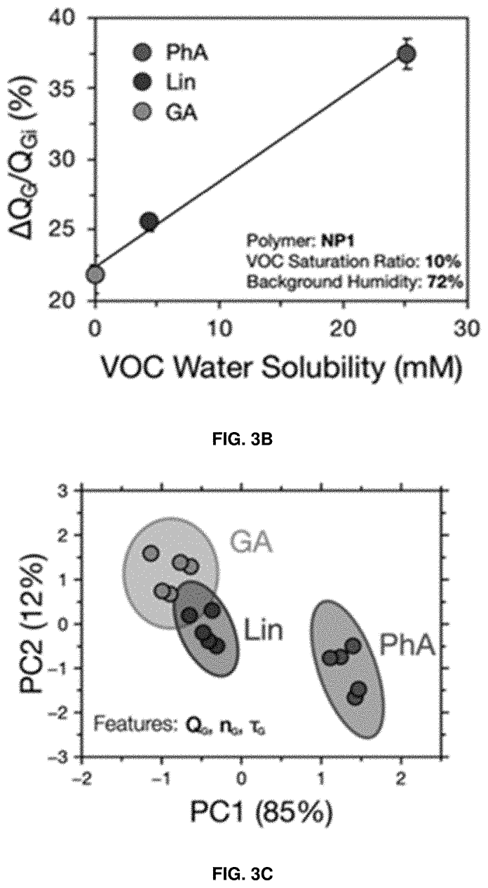

The result suggests that Type I sensor response derives principally from VOC interactions with the water layer. VOC sorption with the water layer can cause vapor pressure lowering that stimulates additional water from the vapor phase to incorporate within the water layer (FIG. 3A). See, for example, K. S. Alexander, et al., J. Stat. Phys. 2005, 119, 479, which is incorporated by reference in its entirety. The incorporation of additional water within the water layer results in a detectable change in water layer impedance, a Type I sensor response. Correspondingly, Type I sensor response is expected to increase with VOC solubility with water. So, PhA is expected to have the highest Type I response followed by Lin and then GA. See, for example, US EPA, Estimation Program Interface (EPI) Suite, United States Environmental Protection Agency, Washington, D.C., 2020, which is incorporated by reference in its entirety. To test this hypothesis, a Type I sensor (fs/2 nm NP1) was exposed to each of the HLB VOCs at an equivalent saturation ratio (SR), a thermodynamic quantity that relates the ratio of a molecule's vapor pressure to its saturation pressure (SR=p/pₛₐₜ). A SR of 10% was employed for this test, which corresponds to PhA, Lin, and GA vapor phase concentrations of 50 ppm, 20 ppm, and 2 ppm, respectively. Results of these tests confirm the expected positive relationship in Type I sensor response with VOC-water solubility (FIG. 3B) providing support for the proposed Type I sensing mechanism.

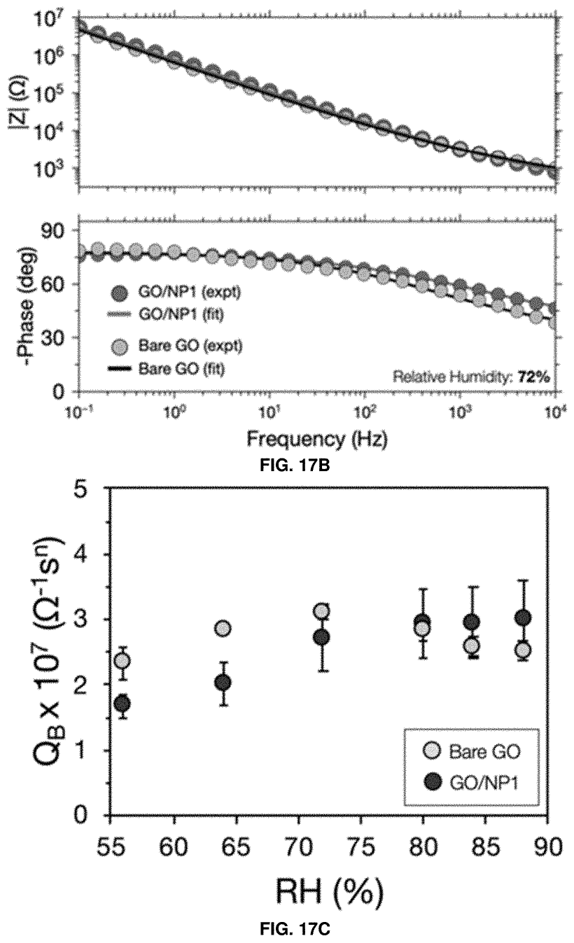

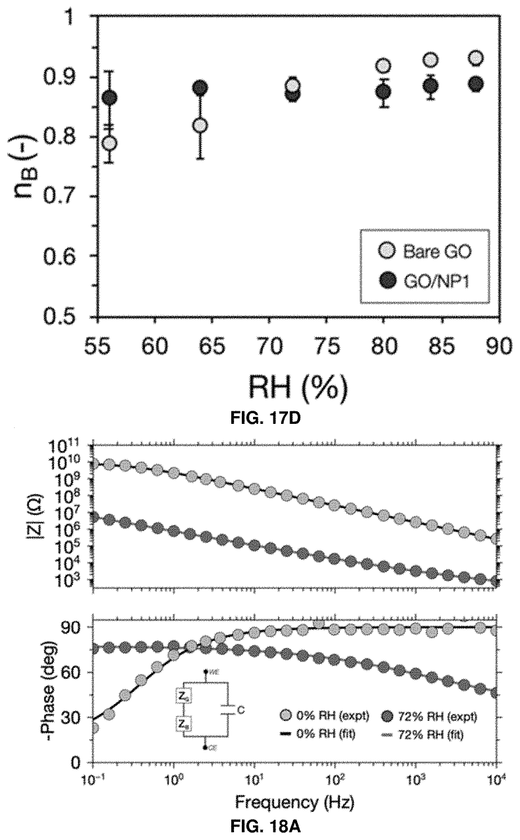

Next, three features extracted from these experiments were combined—response in Q_{G}, response in n_{G}, and τ_{G} (FIGS. 17A-17D)—to perform principal component analysis (PCA) (FIG. 3C). It was found that that PhA can readily be distinguished from GA and Lin, which can be attributed to its comparatively high solubility in water. GA and Lin cannot be fully distinguished from one another, which we attribute to GA and Lin both having similarly low water solubility. However, it should be noted that it is possible to distinguish GA, Lin, and PhA when the SR is doubled to 20% (FIGS. 16A-16C).

In summary, Type I sensors were successfully constructed and their sensing capabilities was demonstrated. Type I sensors were found to be significantly more stable than bare fs and so, stability is a primary advantage of Type I sensors relative to bare fs. Additionally, it was found that Type I sensor response is not significantly influenced by the particular chemistry of the ultrathin iCVD polymer coating. Rather, strong evidence was found that VOC-water solubility is a principal driver of Type I sensor response.

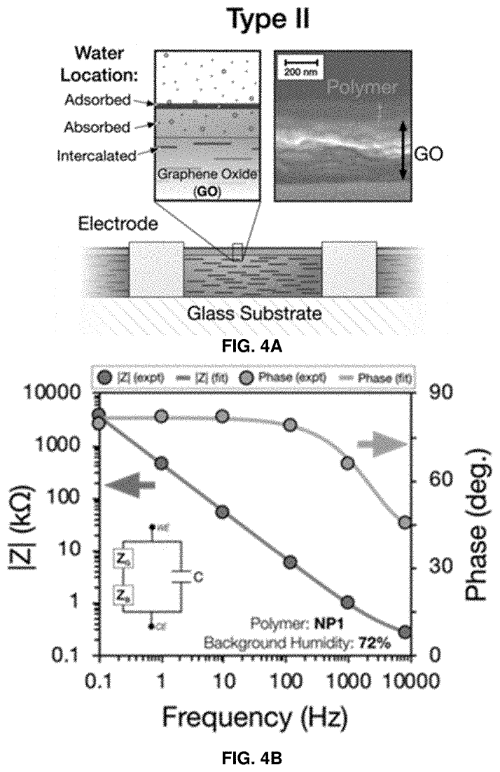

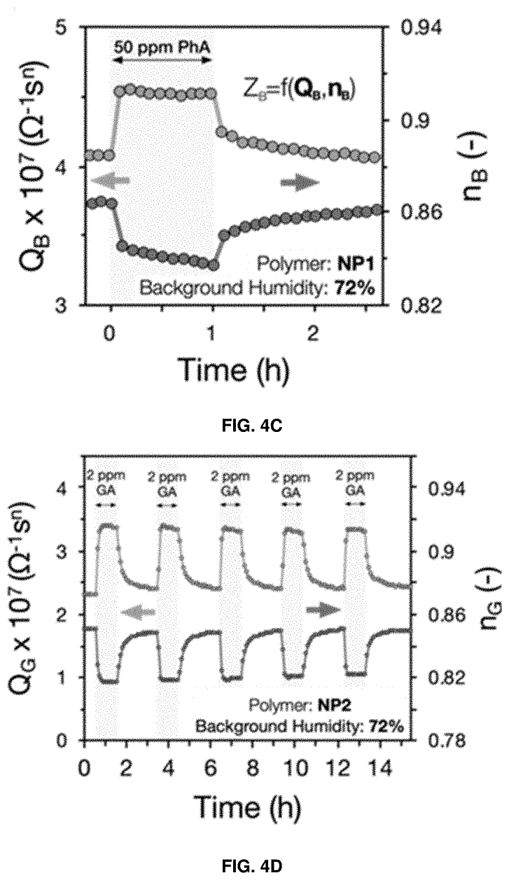

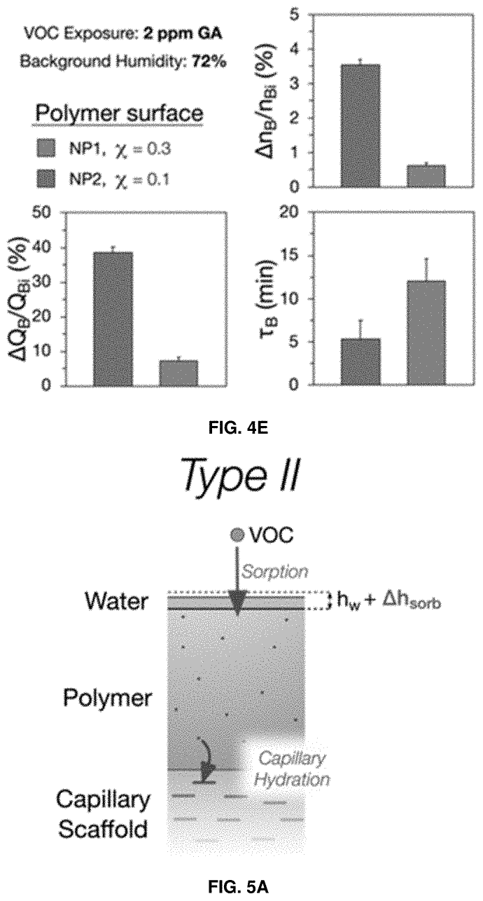

In humid environments, a Type II HIG sensor is comprised of three water-rich regions (FIG. 4A, top left). From top to bottom, these water-rich regions include: an adsorbed water layer (akin to Type I sensors) that can sorb water and VOCs, a polymer film that also sorbs water and VOCs, and a capillary-forming material that intercalates water but not VOCs. For the latter material, films of graphene oxide (GO) can be employed as an example. GO films are composed of stacks of individual GO sheets that form capillaries at sheet-sheet interfaces. See, for example, L. Chen, et al., Nature 2017, 550, 1, which is incorporated by reference in its entirety. These capillaries are hydrophilic and thus take up water easily. However, GO capillaries are highly impermeable to molecules other than water. See, for example, R. K. Joshi, et al., Science (80). 2014, 343, 752; R. Nair, et al. Science (80). 2012, 335, 442; and K. H. Thebo, X. Qian, Q. Zhang, L. Chen, H. M. Cheng, W. Ren, Nat. Commun. 2018, 9, 1, each of which is incorporated by reference in its entirety. Notably, GO films demonstrate negligible permeance of organic molecules, including benzoic acid and toluene, over more than 10 days of study. See, for example, R. K. Joshi, et al., Science (80). 2014, 343, 752, which is incorporated by reference in its entirety. Based on these previous observations, vapor likely cannot penetrate into GO capillaries during an iCVD deposition and thus, iCVD polymers do not deposit within GO capillaries. Rather, an iCVD polymer deposits as a blanket layer atop a GO film. This effect would enable the stacked architecture of a Type II HIG sensor.

This hypothesis is supported by three key observations. First, we perform vacuum adsorption measurements with QCM using an iCVD monomer adsorbate (butyl acrylate) (FIGS. 9A-9C), and observe that a GO-coated QCM crystal does not provide a measurable surface area enhancement relative to an uncoated QCM crystal (Table 4). This result indicates that iCVD monomer permeation into GO capillaries is negligible. Second, NP1 mass deposited on GO-coated and bare QCM crystals under the same iCVD conditions were compared and it was observed that the deposited mass is nearly identical (<10% different) (FIG. 10). This result supports the hypothesis, since if iCVD polymer did penetrate appreciably into GO film capillaries, one would measure significantly increased mass of deposited NP1 on the GO-coated QCM crystal. Finally, SEM was employed to image a GO film cross-section after NP4 deposition, and observe a GO film coated in a blanket layer of NP4 (FIG. 4A, top right). Thus, an iCVD polymer deposits as a blanket layer atop a GO film with negligible polymer penetration into capillaries. IDEs coated in GO and then subsequently coated in nanoscale (<30 nm) iCVD polymer as Type II sensors.

Next, the impedance characteristics of bare GO films and Type II sensors were explored. When exposed to humidity, a bare GO film takes in water from the vapor phase into its capillaries, and impedance introduced by this capillary-intercalated water dominates the GO film impedance spectrum. See, for example, H. Bi, et al., Sci. Rep. 2013, 3, 1, which is incorporated by reference in its entirety. Thus, GO film impedance is well approximated by a circuit model composed of a capacitor in parallel with series Z_{G} and Z_{B}, which represent contributions from GTR and bulk-like impedance behavior of capillary-intercalated water, respectively. Note that this same circuit is used to model Type I impedance and perform Type I VOCs sensing (FIGS. 2A-2E). Spectra were collected across a wide range of high RH (55-90%) for bare GO and a Type II sensor composed of 25 nm PBA (polybutylacrylate), and excellent fits were achieved across the 100 mHz-10 kHz test frequency range (FIGS. 15A-15D). From these spectra and fits, two observations were made. First, Q_{B} and n_{B} (which comprise Z_{B}) are nearly identical across the tested humidity range for bare GO and Type II sensors. This result indicates that the blanket layer of iCVD polymer is not a significant barrier to water transport from the vapor phase into GO capillaries and also that the blanket polymer layer does not provide a substantial impedance contribution. Second, Z_{B} is the most visible impedance feature for Type II sensors at high RH (FIG. 4B), while ZG is the most visible impedance feature for Type I sensors at high RH (FIG. 2B). This distinction is due to a difference in the type of water probed: Type II sensor impedance derives from capillary-intercalated water, while Type I impedance derives from a thin water layer. Thus, Type II sensor impedance spectra at high RH derives principally from water contained within GO capillaries, as it does for bare GO films. All subsequent tests were performed at 72% RH to remain consistent with the Type I sensors.

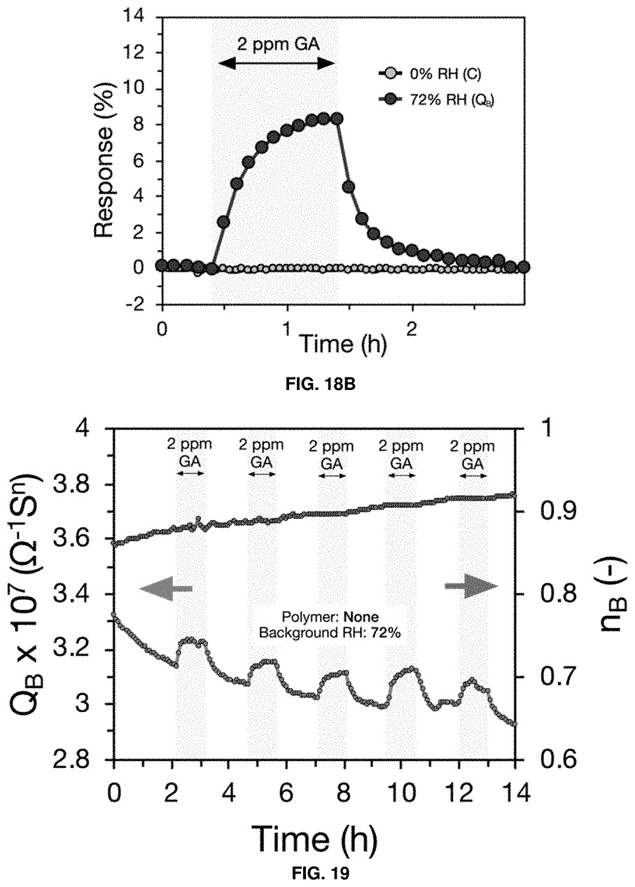

Next, a Type II sensor composed of 26 nm NP1 (GO/NP1) was prepared and its VOCs sensing capabilities were explored. To do this, the same transient impedance spectra collection and fitting process was used as we applied to Type I sensors. Response in both Q_{B} and n_{B} was observed for GO/NP1 during a to 50 ppm PhA exposure experiment at 72% background RH (FIG. 4C). Thus, Type II sensors can be used to detect VOCs. Moreover, since Type II sensors do not yield response to VOCs at 0% RH but do yield response at 72% RH (FIGS. 18A-18B), we classify Type II sensors as HIG sensors.

Type II sensor stability by testing over long time scales was evaluated. Specifically, a Type II sensor composed of 16 nm NP2 was tested over a 14 h period including five 1 h exposures to 2 ppm GA at background RH (FIG. 4D). From this test, limited response decay in Q_{B} (7%) and n_{B} (0.1%) was observed. Furthermore, limited background drift in Q_{B} (5%) and n_{B} (1%) was observed. Stability of Type II sensors can be superior to that of bare GO (FIG. 19 and Table 6), but the observed improvement is not as significant as previously observed for Type I sensors relative to bare fs (Table 5). Type I and Type II sensors both demonstrate similarly high stability.

Next, the effects of polymer selection on Type II sensor performance were evaluated. To do this, Type II sensors incorporating either NP1 (χ=0.29) or NP2 (χ=0.11) were prepared and their when exposed to 2 ppm GA at 72% background RH was measured (FIG. 4E). Q_{B} and n_{B} response of GO/NP2 was 5× higher than that of GO/NP1. Furthermore, Z_{B} response time was defined as τB, the 1/e time constant for Q_{B}, and τ_{B} for GO/NP1 was observed to be 2× higher than that for GO/NP2. These results contrast with the results of identical tests performed on Type I sensors (FIG. 2E) that indicate that polymer chemistry does not significantly affect Type I response or response time. Thus, unlike Type I sensors, Type II sensor performance can be tuned significantly by polymer selection.

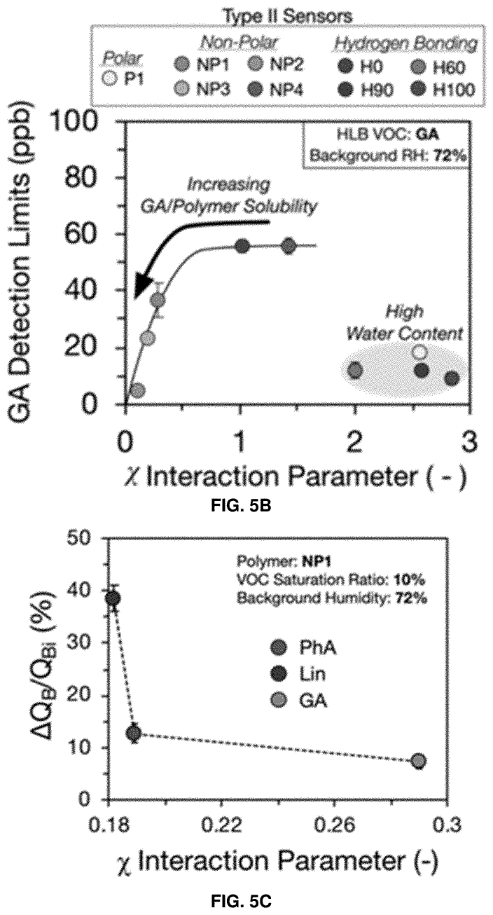

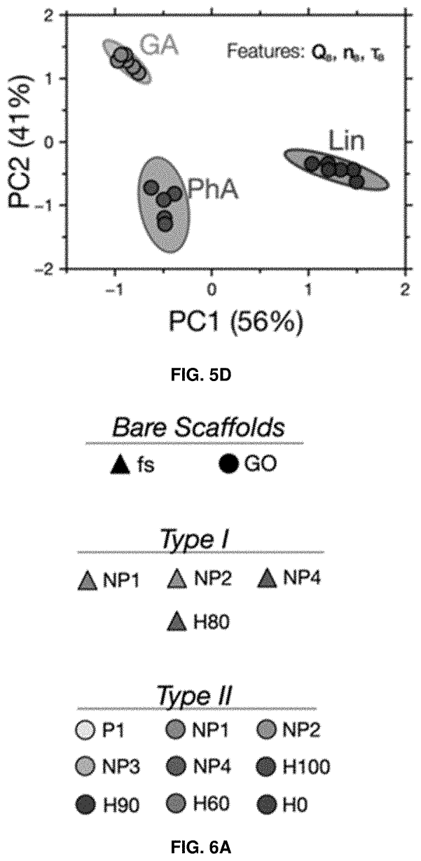

This result suggests that Type II sensor response derives principally from VOC interactions with the polymer layer (FIG. 5A). Without being bound to any particular theory of operation, it appears that VOC sorption with the polymer layer of a Type II sensor displaces water, resulting in net water entry into the water capillary scaffold. The incorporation of additional water within capillaries results in a measurable change in capillary water impedance, a Type II sensor response. Note that VOCs are not expected to penetrate appreciably into the GO film, since GO films exhibit near-zero permeability to organic molecules, a characteristic preserved for small organic molecules such as ethanol in the presence of water vapor. See, for example, R. K. Joshi, et al., Science (80). 2014, 343, 752; R. Nair, et al. Science (80). 2012, 335, 442; and K. H. Thebo, et al., Nat. Commun. 2018, 9, 1, each of which is incorporated by reference in its entirety. Thus, Type II sensor response can be expected to increase with the degree of polymer-VOC solubility (lower χ). The much larger response observed for lower χ GO/NP2 relative to higher χ GO/NP1 is support for the proposed mechanism.

To further explore the effects of polymer chemistry on Type II response, a plot of GA detection limit vs polymer-GA χ interaction parameter was prepared for 9 different Type II HIG sensors composed of non-polar, polar, and hydrogen bonding polymers (FIG. 5B). All tests consisted of at least five GA exposures at 10% SR and 72% background RH. Detection limits were then calculated from changes in Q_{B} assuming a cutoff for detection at a signal-to-noise ratio (SNR) of 3 using the following relationship:

$$Detection Limit = \frac{C {σ}_{b}}{Δ S} ( {SNR}_{c} ) \qquad ( 4 )$$

(S. Vaddiraju, K. K. Gleason, Nanotechnology 2010, 21, which is incorporated by reference in its entirety)

where C is the concentration of VOC in the vapor phase during the test (2 ppm), SNR_{c} is the SNR cutoff value (3), σ_{b} is the measured background noise as expressed as a standard deviation (Ω⁻¹sⁿ), and ΔS is the measured change in signal when exposed to VOC (Ω⁻¹sⁿ). Several trends were observed in the prepared plot. First, for non-polar acrylate polymers with χ<0.5 (NP1, NP2, and NP3), a positive relationship between χ and GA detection limits was observed. This result is consistent with the observation that Type II sensor response is positively correlated to VOC-polymer solubility (FIG. 4E). Second, fully crosslinked polymers (NP4 and H0) have high GA detection limits of ˜55 ppb. Since GA has a much larger diameter (0.9 nm) than both the mesh size of H0 (<0.5 nm) (J. L. Yagüe, K. K. Gleason, Soft Matter 2012, 8, 2890, which is incorporated by reference in its entirety) and the ring diameter of NP4 (0.4 nm), (B. H. Shen, S. Wang, W. E. Tenhaeff, Sci. Adv. 2019, 5, 1, which is incorporated by reference in its entirety) it is proposed that these relatively high detection limits are the result of size exclusion; GA cannot absorb appreciably within these fully crosslinked polymers and so, response of these Type II sensors is limited to that of other sorption processes. Finally, polar (P1) and hydrogen bonding (H60, H90, H100) polymers with mesh size above GA size exclusion have very low solubility for GA (χ>2) and yet have low GA detection limits (<20 ppb). This result can be attributed to the higher water content of these polymers relative to non-polar polymers. For example, at high RH above 80%, the water content of H100 and other hydrophilic acrylate polymers is >30% v/v while that of non-polar acrylate polymers is <2.5% v/v, a difference of more than ten-fold. See, for example, K. Unger, et al., Macromol. Chem. Phys. 2016, 217, 2372; and W. L. Chen, et al., Macromolecules 1999, 32, 136, each of which is incorporated by reference in its entirety. Consistent with the proposed mechanism (FIG. 5A), increased water content of a Type II sensor polymer would increase water displacement in the polymer per VOC sorption event to stimulate a larger Type II response. Thus, two routes can be identified to lower detection limits with Type II sensors—1) increase VOC-polymer solubility by decreasing χ, or 2) increase water content of polymer by selecting a polar or hydrogen bonding polymer.

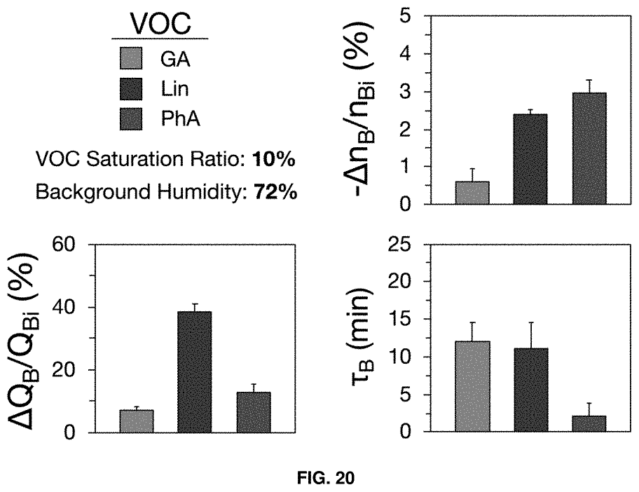

To further evaluate the proposed Type II sensor mechanism, the relative response of a single Type II sensor composed of a non-polar polymer (NP1) to the three HLB VOCs was assessed. The χ values for PhA-NP1, Lin-NP1, and GA-NP1 were estimated to be 0.19, 0.18, and 0.29 (Equation 2). Thus, if VOC-polymer solubility underpins Type II sensor response, we would expect Lin to provide the highest response followed by PhA and then GA. Note that this ordering differs from that predicted and observed for Type I response (FIG. 3B). In tests identical to those conducted for a Type I sensor (FIG. 3B), the expected positive relationship in Type II sensor response with polymer-VOC solubility was observed (FIG. 5C). Response in Q_{B}, response in n_{B}, and τ_{B} were extracted from these experiments (FIG. 20) as features for principle component analysis (PCA) and it was found that a GO/NP1 Type II sensor can readily distinguish PhA, Lin, and GA at 10% SR (FIG. 5D). This result contrasts with the incomplete classification of the HLB VOCs at 10% SR achieved by a fs/NP1 Type I sensor (FIG. 3C).

In summary, Type II sensors were successfully constructed, their impedance characteristics explored, and their sensing capabilities demonstrated. Like Type I sensors, Type II sensors are highly stable. However, unlike Type I sensor performance, Type II sensor performance can be significantly tuned by polymer selection.

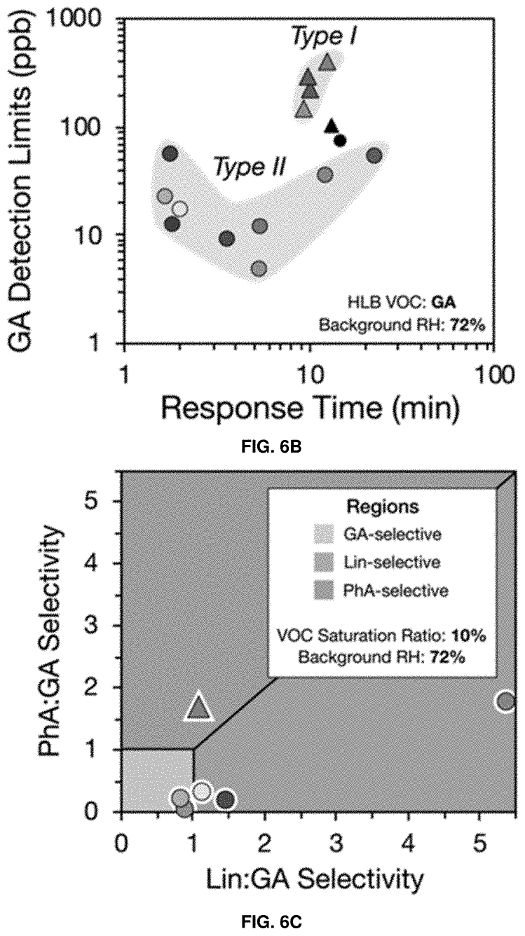

Four HIG Type I, 10 HIG Type II, and also test the 2 bare scaffolds were prepared to evaluate and compare the effects of sensor architecture on sensor figures of merit (FOMs), which include detection limits, response time, and selectivity (FIGS. 6A-6D). The overall results of these tests are also summarized in table form (Table 1).

For each test, the sensor was exposed to a selected HLB VOC at 10% SR with a background RH of 72%. Impedance spectra were measured from 100 mHz to 10 kHz. For Type I and bare fs, Z_{G} is most visible across the test frequency range and so, Z_{G} response and response times are used in our analysis of these sensors. For Type II and bare GO, Z_{B} is most visible across the test frequency range and so, Z_{B} response and response times are used in our analysis of these sensors. Discussion is limited to Q_{G} and Q_{B} FOMs as they are observed to have lower detection limits than n_{G} and n_{B}, respectively. Thus, Q denotes either Q_{G} or Q_{B} depending on whether the sensor employs a fs or GO scaffold, respectively. Detection limits for Q were calculated using Equation (4). Response times are presented as the 1/e time constant for Q. Selectivities are expressed as a Q response ratio of GA to Lin or GA to PhA.

A log-log plot of GA detection limits vs response time was prepared for sensors tested with 10% SR (2 ppm) GA at 72% background RH (FIG. 6A). GA was selected for this analysis since GA isbthe HLB VOC most implicated in the onset of asymptomatic HLB. See, for example, A. A. Aksenov, et al., Anal. Chem. 2014, 86, 2481, which is incorporated by reference in its entirety. The prepared detection limit vs response time plot revealed differences between and within sensor categories.

To begin the analysis, Type I and Type II sensors were compared. Significant advantages of Type II relative to Type I sensors were observed in both detection limit and response time (FIG. 6A). First, Type I sensors have significantly higher detection limit than Type II sensors. For example, a Type I sensor composed of NP2 has a detection limit of 150 ppb while a Type II sensor composed of NP2 has a detection limit of 5 ppb, a difference of 30-fold. Second, Type II sensors often have significantly lower response time than Type I sensors. For instance, a Type I sensor composed of NP2 has a response time of 9 min while a Type II sensor composed of NP2 has a response time of 5 min, a 40% reduction. Moreover, the fastest response time recorded for Type I sensors is 5× that of Type II sensors. Therefore, Type II sensors can be considered superior to Type I sensors in both detection limits and response time for certain applications.

Differences within sensor categories were compared, and the initial discussion relates to Type I sensors (FIG. 6A). First, coating fs in ultrathin polymer to create a Type I HIG sensor results in a slight decrease in response time (5-30% reduction) but a more significant increase in detection limit (>60% increase). Since it was additionally observed that Type I sensors provide significant stability improvements relative to bare fs (Table 5), a trade-off was identified between lower detection limits (provided by bare fs) and higher stability (provided by a Type I sensor). Next, mild variability between Type I sensors in detection limits (150-410 ppb) and limited variability in response time (9-13 min) was observed despite the different polymer chemistries employed (0.1<χ<2.6). Thus, Type I sensors are not a highly tunable sub-class of HIG sensors.

Next, Type II sensors are discussed (FIG. 6A). Coating GO in iCVD polymer to form a Type II HIG sensor has significant beneficial effects on detection limit and response time. For example, coating GO in NP3 results in a more than 7-fold reduction in response time (14 min to 2 min) and a 65% reduction in detection limit (66 ppb to 23 ppb). Next, high variability was observed in Type II sensor detection limits based on the polymer selected (5-55 ppb). These results have already been discussed previously (FIG. 5B). Finally, high variability between Type II sensor response time was observed depending on the polymer chosen (2-22 min). Note that the minimum cycle time employed for impedance tests is ˜3 min and so, one can interpolate 1/e response time of transient responses that reach a final value before cycle time completion to be approximately 2 min. This estimate was applied to GO/NP3, GO/H90, and GO/H0 responses. Thus, in these cases, the true response time is very likely below 2 min.

Hydrogel crosslinking was also observed to affect Type II HIG sensor response time (FIG. 6A). For example, lightly crosslinked hydrogel (H90) neither lowers mesh size to size-exclusionary range for GA nor affects solubility significantly (both have χ>2.5). See, for example, J. L. Yagüe, K. K. Gleason, Soft Matter 2012, 8, 2890, which is incorporated by reference in its entirety. However, this light crosslinking is observed to halve response time relative to non-crosslinked H100 (4 min to 2 min), while incurring only a minor penalty in detection limits (9 ppb to 12 ppb). The observed faster response time with light crosslinking can be attributed to increased rate of water diffusion within the hydrogel layer. However, further crosslinking, as illustrated by H60, can reverse this trend and result in increased response time. The wide spectrum of positions of Type II sensors within the response time vs detection limits plot clearly illustrates that polymer chemistry strongly affects Type II performance and thus, Type II sensors are a highly tunable class of VOCs sensors.

Finally, a 2-D selectivity plot for a select portion of Type I (polymer: NP1) and Type II (polymer: P1, NP1, NP2, NP3, H100) sensors was prepared (FIG. 6b). Location within a particular region of this selectivity plot indicates whether a HIG sensor is most responsive to, and thus selective for, GA (green region), Lin (red region), or PhA (blue region). The HIG sensors studied span each of the three regions and so, HIG sensors can be constructed to be selective for each of the HLB VOCs.

In addition, complementary selectivity for Type I and Type II sensors was observed (FIG. 6B). Type I sensors are selective for PhA. Type II sensors can be selective for either Lin or GA depending on the polymer employed. However, Type II sensors do not demonstrate PhA-selectivity with any of the polymers employed. The lack of PhA-selectivity of Type II sensors can be attributed to the strong affinity of PhA to the thin water layer that resides above the iCVD polymer film. In a Type I sensor, PhA sorption with the thin water layer results in a significant response, because the sensing signal derives from the impedance of the thin water layer. However, Type II sensor signal derives from the impedance of water intercalated within GO film capillaries. Thus, for Type II sensors, VOCs sorption with the thin water layer does not result in a large response, resulting in low PhA-selectivity for Type II sensors and the observed complementary selectivities for Type I and Type II sensors.

In conclusion, the HIG sensing concept is conceived and demonstrated herein, with which VOCs are detected by monitoring the impedance of water-rich regions that form at sensor interfaces in humid environments. Two HIG sensor variants have been constructed, namely Type I and Type II, which incorporate different scaffold materials that are coated by nanoscale iCVD polymer. Type I sensors incorporate a high surface area fs nanoparticle film scaffold, and there is evidence that Type I sensing derives from the impedance of a thin water layer formed atop the polymer-coated scaffold. Type II sensors incorporate a GO film scaffold, and there is evidence that Type II sensing derives from the impedance of water intercalated within GO capillaries.

Type I and Type II sensor performance was evaluated in detecting three VOCs implicated in HLB. It was found that both Type I and Type II sensors demonstrate good stability in terms of background drift and response decay during testing over long time scales. However, it was found that Type I and Type II sensors respond to VOCs differently. Type I sensors response is not significantly affected by the selection of iCVD polymer, but rather is well-correlated with water-VOC solubility. In contrast, Type II sensor response is significantly affected by the selection of iCVD polymer, with larger responses achieved with increasing polymer-VOC solubility. Thus, Type II sensor performance can be tuned significantly by polymer selection. Additionally, it was found that Type II sensors are superior to Type I sensors in terms of key FOMs, including response time and sensitivity. Notably, best-in-class Type II sensors achieve <2 min response time and <10 ppb detection limit for GA. Furthermore, HIG sensors were constructed that are selective for each of the HLB VOCs, and find that Type I and Type II sensors have complementary selectivity. Finally, it was found that HIG sensors address the key requirements of remote field detection of VOCs and so, have significant potential as VOC sensors for crop disease detection and other high impact applications.

Experimental Section

HIG Sensor Scaffold Preparation: 10 μL of fs or GO particle solutions were drop cast atop IDEs (Micrux, ED-IDE3-Au) to form scaffolds for Type I or Type II HIG sensors, respectively. During drop casting, IDEs were placed on a hot plate at controlled temperature of 60° C. To prepare fs particle solutions used in drop casting, 8 mg of fs (Aldrich) was mixed with DI water and sonicated for 10 min. Two equential 10 μL drop cast steps were employed to prepare fs scaffolds. To prepare GO solutions used in drop casting, a 1 g/L GO solution with 90-200 nm GO flake size (Graphene Supermarket) was diluted with DI water at a GO solution to DI water ratio of 1:3. A single 10 μL drop cast step was employed to prepare GO scaffolds. Scaffold surface area was measured using adsorption experiments by modifying a previously described procedure. See, for example, K. K. S. Lau, K. K. Gleason, Macromolecules 2006, 39, 3688, which is incorporated by reference in its entirety. Further details on adsorption measurements and surface area calculations are provided below.

Polymer Synthesis: Polymer films were synthesized by iCVD using a previously described setup and procedure. See, for example, X. Wang, et al., ACS Sensors 2016, 1, 374, which is incorporated by reference in its entirety. Briefly, tert-butyl peroxide initiator along with monomer and other flows (nitrogen, crosslinker) were drawn through a custom built iCVD reactor. Filament temperature of 250° C. was achieved by passing 1.2 A through a Nichrome filament array at 1.5 cm distance from the substrate. Film thickness was monitored with in-situ interferometry with a 633-nm HeNe laser. Setpoints used in all iCVD depositions are provided in Table 3.

Polymer Characterization: The thickness of iCVD polymer films used in each HIG sensor was measured using variable angle spectroscopic ellipsometry (VASE, J. A. Woollam Model M-2000). Three incident angles—65°, 70°, and 75°—were incorporated in each individual film thickness measurement. Data from ellipsometry experiments were fit with a Cauchy-Urbach model to determine polymer thickness. Ellipsometry measurements were performed on silicon (Si) substrates that were coated by iCVD along with the corresponding sensor samples. FTIR measurements were performed on hydrogel films that were deposited on Si substrates. For these measurements, we used a Nicolet Nexus 870 spectrometer with a DTGS KBr detector in normal transmission mode averaged over 64 scans. The measurement range was 400 to 4000 cm⁻¹ and the resolution was 4 cm⁻¹. All spectra used in composition calculations (FIG. 8) were background subtracted and baselined. Additionally, all samples used in FTIR measurements were dried overnight at 60° C. in a vacuum oven.

Sensor Characterization: VOCs sensing was performed in a custom-built gas flow cell connected to a CHI660 potentiostat (CH Instruments). EIS measurements were performed continuously from 100 mHz to 10 kHz with a 50 mV voltage amplitude and fit to a circuit model using MATLAB. Three gas flows were produced using programmable mass flow controllers (Alicat) and mixed to prepare a combined flow to the testing cell at a specified RH and VOC SR. The flows consisted of a nitrogen flow (F1), sparged flow through a water bubbler (F2), and sparged flow through the liquid of a particular VOC (F3). Total flow rate was a constant 2 slpm throughout the duration of sensing experiments. F1 and F2 together were used to set a test humidity that remained constant throughout the experiment (72% RH). F3 was used to provide VOCs at specified SR during a programmed exposure event. As has been done previously,^{[32]} we assume F3 contains the VOC at saturation and so, SR is determined by calculating F3 as a percent of total flow (F1+F2+F3=2 slpm). During a sensing experiment, 4 h of flow at 72% RH with no VOCs flow (F3=0) was followed by a series of VOC exposure cycles. A VOC exposure cycle consisted of 1 h of flow at 72% RH and a specified VOC SR followed by at least 1 h of flow at 72% RH and no VOCs flow (F3=0). RH was measured during experiments using a BME280 sensor (Bosch) placed within the gas sensing chamber that also contained HIG sensors.

TABLE 1
Summary of HIG sensors testing results.

| | | | | Polymer | Detection | Response | Selectivity | Selectivity |
|---|---|---|---|---|---|---|---|---|
| | iCVD | | | Thickness | Limits | Time | (Lin:GA), | (PhA:GA), |
| Category | Polymer* | Pendant Chemistry | χ [−] | [nm] | [ppb] | [min] | [−] | [−] |
| Type I | NP1 | 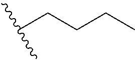 [SMILES: *CCCC] | 0.3 | 2 | 413 ± 32 | 12.5 ± 4.0 | 1.2 | 1.7 |
| | NP2 | 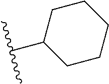 [SMILES: *C1CCCCC1] | 0.1 | 2 | 151 ± 9 | 9.2 ± 2.2 | — | — |
| | NP4 | 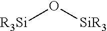 | 1.4 | 2 | 227 ± 15 | 9.9 ± 2.9 | — | — |
| | H80 | 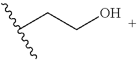 [SMILES: *CCO] | 2.4 | 9 | 292 ± 25 | 9.8 ± 3.8 | — | — |
| | | 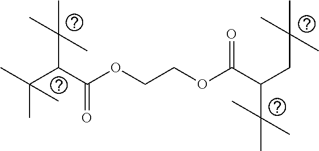 [SMILES: CC(C)(C)CC(C(=O)OCCOC(=O)C(C(C)(C)C)C(C)(C)C)C(C)(C)C] | | | | | | |
| Type II | P1 | 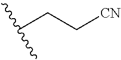 [SMILES: *CCC#N] | 2.6 | 20 | 18 ± 2 | 2.0 ± 0.8 | 1.1 | 0.3 |
| | | 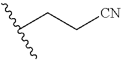 [SMILES: *CCC#N] | | | | | | |
| | NP1 | 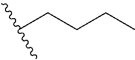 [SMILES: *CCCC] | 0.3 | 26 | 37 ± 6 | 12.0 ± 2.6 | 5.4 | 1.8 |
| | NP2 | 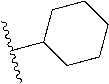 [SMILES: *C1CCCCC1] | 0.1 | 16 | 5 ± 1 | 5.3 ± 2.2 | 0.9 | 0.1 |
| | NP3 | 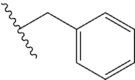 [SMILES: *Cc1ccccc1] | 0.2 | 25 | 23 ± 1 | 1.7 ± 0.5 | 0.8 | 0.2 |
| | NP4 | 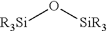 | 1.4 | 7 | 56 ± 3 | 22.0 ± 2.7 | — | — |
| | H100 | 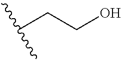 [SMILES: *CCO] | 2.9 | 21 | 9 ± 2 | 3.6 ± 0.8 | 1.5 | 0.2 |
| | H90 | 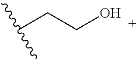 [SMILES: *CCO] | 2.6 | 24 | 12 ± 1 | 1.8 ± 1.1 | 1.2 | 0.2 |
| | | 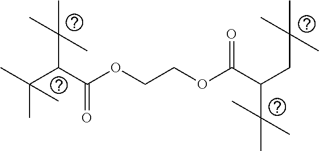 [SMILES: CC(C)(C)CC(C(=O)OCCOC(=O)C(C(C)(C)C)C(C)(C)C)C(C)(C)C] | | | | | | |
| | H60 | 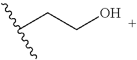 [SMILES: *CCO] | 1.9 | 27 | 12 ± 3 | 5.4 ± 1.2 | 1.5 | 0.1 |
| | | 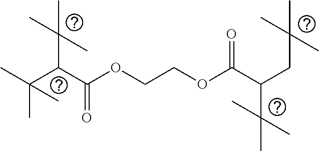 [SMILES: CC(C)(C)CC(C(=O)OCCOC(=O)C(C(C)(C)C)C(C)(C)C)C(C)(C)C] | | | | | | |
| | H0 | 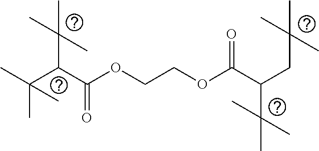 [SMILES: CC(C)(C)CC(C(=O)OCCOC(=O)C(C(C)(C)C)C(C)(C)C)C(C)(C)C] | 1.0 | 20 | 56 ± 2 | 1.8 ± 0.1 | 1.4 | 0.2 |

*NP = non-polar, H = hydrogen bonding, P = polar

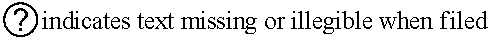

A VOCs sensor concept is demonstrated herein based on the impedance of water assembled at sensor interfaces when exposed to humidity, what is referred to herein as Humidity-Initiated Gas (HIG) sensors. Two HIG sensor variants are described—Type I and Type II—that have different sensing characteristics. HIG sensors represent an attractive alternative to existing VOCs sensors for remote field detection applications

Remote Field Detection Scoring Criteria for VOCs Sensing Technologies

Different sensors technologies were scored according to the below criteria. Scores for canine, metal oxide semiconductor (MOS), and mass spectrometry were assigned based on reference to the literature. Scores for HIG sensors were assigned based on the results of this the manuscript. All scores are also available in Table 6, and are visualized in FIG. 1C.

### 1. High Sensitivity

Score=5: <100 ppt
Score=3: 10 ppb
Score=1: 1 ppm

### 2. Tunability

Score=5: Single sensors architecture permits wide tunability to suit application
Score=3: Limited tunability possible with same sensors architecture
Score=1: Entire sensors architecture must be changed to tune sensors

### 3. Fast Response

Score=5: <10 s
Score=3: 10 min
Score=1: >1 h
   4. Small Size (largest dimension)
Score=5: <1 cm
Score=3: 10 cm
Score=1: >1 m

### 5. Response Interpretability

Score=5: Good fundamental understanding of sensing mechanism
Score=3: Phenomenological understanding for how sensing mechanism occurs
Score=1: ‘Blackbox’ understanding for how sensing mechanism occurs

### 6. Humidity Resilience

Score=5: Performance is enhanced by humidity above 60%
Score=3: Mild reduction in performance at humidity above 60%
Score=1: Significant reduction in performance at humidity above 60%

TABLE 2
Scores for sensors technologies according to remote field detection requirements

| | High | High | Fast | Small | Interpretable | Humidity |
|---|---|---|---|---|---|---|
| Sensor | Tunability | Sensitivity | Response | Size | Response | Resilience |
| HIG Type I | 3 | 2 | 3 | 5 | 5 | 5 |
| HIG Type II | 5 | 4 | 4 | 5 | 4 | 5 |
| Canine^{[1-3]} | 5 | 5 | 5 | 1 | 1 | 5 |
| MOS^{[4, 5}] | 3 | 4 | 4 | 5 | 4 | 2 |
| Mass Spectrometry^{[6, 7]} | 5 | 5 | 2 | 1 | 5 | 4 |

TABLE 3
Recipes for iCVD depositions

| | P_{chamber} | T_{stage} | F_{N2} | F_{TBPO} | Fₓₗ [sccm] | Fₘₒₙ [sccm] | SRₓₗ | SRₘₒₙ |
|---|---|---|---|---|---|---|---|---|
| Polymer | [mTorr] | [° C.] | [sccm] | [sccm] | Acronym [—] | Acronym [—] | [—] | [—] |
| P1 | 200 | 40 | 0 | 0.6 | — | 0.1 | — | 0.22 |
| | | | | | | CEA | | |
| NP1 | 1000 | 23 | 3.0 | 0.6 | — | 0.2 | — | 0.01 |
| | | | | | | BA | | |
| NP2 | 1000 | 40 | 2.0 | 1.0 | — | 0.2 | — | 0.10 |
| | | | | | | CHMA | | |
| NP3 | 100 | 40 | 0 | 0.6 | — | 0.1 | — | 0.12 |
| | | | | | | BMA | | |
| NP4 | 225 | 40 | 0 | 0.9 | — | V4D4 | — | 0.12 |
| H0 | 100 | 40 | 1.8 | 0.6 | 0.6 | 0 | 0.07 | 0 |
| | | | | | EGDA | HEMA | | |
| H60 | 100 | 40 | 1.3 | 0.6 | 0.6 | 0.5 | 0.07 | 0.03 |
| | | | | | EGDA | HEMA | | |
| H80 | 200 | 40 | 0.4 | 1.0 | 0.1 | 0.5 | 0.04 | 0.10 |
| | | | | | EGDA | HEMA | | |
| H90 | 100 | 40 | 0.75 | 0.6 | 0.15 | 1.5 | 0.02 | 0.10 |
| | | | | | EGDA | HEMA | | |
| H100 | 200 | 40 | 0.5 | 1.0 | 0 | 0.5 | 0 | 0.10 |
| | | | | | EGDA | HEMA | | |

^{a)} Acronyms: EGDA (ethylene glycol diacrylate), CEA (cyanoethyl acrylate), BA (butyl acrylate), CHMA (cyclohexyl methacrylate), BMA (benzyl methacrylate), V4D4 (1,3,5,7- tetravinyl-1,3,5,7-tetramethylcyclotetrasiloxane), HEMA (hydroxyethyl methacrylate).
Polymers are denoted with a P in front of the acronym.

FTIR to Determine EGDA and HEMA Composition in Hydrogels

EGDA and HEMA composition in hydrogels were determined using FTIR measurements as has been described previously. See, for example, K. Chan, K. K. Gleason, Langmuir 2005, 21, 8930; J. L. Yagüe, K. K. Gleason, Soft Matter 2012, 8, 2890; and W. Li, et al., ACS Appl. Mater. Interfaces 2019, 11, 5668, each of which is incorporated by reference in its entirety. Two FTIR absorbance features are used in hydrogel composition determination: (1) C═O stretching from incorporated EGDA and HEMA (1750-1690 cm⁻¹) and (2) O—H stretching from incorporated HEMA (3700-3050 cm⁻¹). The peak area for C═O stretching is denoted A_{C═O} and the peak area for O—H stretching is denoted A_{O—H}. r is defined as the ratio of A_{C═O} to A_{O—H} for a hydrogel film with no incorporated EGDA (100 mol % HEMA). Our measured value for r is 1.46. HEMA concentrations of hydrogel films that also incorporate EGDA were calculated using (W. Li, et al., ACS Appl. Mater. Interfaces 2019, 11, 5668, which is incorporated by reference in its entirety)

$$\frac{[ HEMA ]}{[ HEMA ] + [ EGDA ]} = \frac{{rA}_{O - H}}{{rA}_{O - H} + \frac{1}{2} ( {A}_{C = O} - {rA}_{O - H} )} \qquad ( S1 )$$

Using this method, the HEMA concentration of H90 and H60 was calculated to be 87 mol % and 59 mol %, respectively. Furthermore, the H80 HEMA concentration was estimated by incorporating H90 and H60 information into the mole fraction form of the Mayo-Lewis equation (A. Rudin, P. Choi, The Elements of Polymer Science & Technology, 2013, which is incorporated by reference in its entirety)

$${F}_{A} = \frac{{r}_{A} {f}_{A}^{{′}^{2}} + {f}_{A}^{′} ( 1 - {f}_{A}^{′} )}{{r}_{A} {f}_{A}^{{′}^{2}} + 2 {f}_{A}^{′} ( 1 - {f}_{A}^{′} ) + {{r}_{B} ( 1 - {f}_{A}^{′} )}^{2}} \qquad ( S2 )$$

where f_{A}′ is the HEMA surface mole fraction during deposition, F_{A} is the HEMA mole fraction in the deposited polymer film, r_{A} is the reactivity ratio of HEMA, and r_{B} is the reactivity ratio of EGDA. See, for example, Y. Mao, K. K. Gleason, Langmuir 2006, 22, 1795, which is incorporated by reference in its entirety. A system of equations was solved to derive values for r_{A} (1.49) and r_{B} (0.01), and then used these values to produce an estimate for HEMA concentration of H80 (79 mol %).

BET Analysis of HIG Sensor Scaffolds

To measure HIG sensor scaffold surface area, we perform quartz crystal microbalance (QCM) adsorption measurements with a butyl acrylate adsorbate using a previously described procedure (FIG. 9A). See, for example, K. K. S. Lau, K. K. Gleason, Macromolecules 2006, 39, 3688, which is incorporated by reference in its entirety. Note that butyl acrylate is the iCVD monomer that comprises NP1 (FIG. 9B). Briefly, butyl acrylate is introduced into a vacuum chamber containing a temperature-controlled QCM instrument, which was maintained at 23° C. for all measurements. The saturation ratio (p/pₛₐₜ) of butyl acrylate is modified by changing the partial pressure of butyl acrylate introduced into the vacuum chamber. Butyl acrylate adsorption on the QCM crystal surface as well as the surfaces of scaffold materials (fs or GO) coated on the QCM crystal results in a shift in QCM crystal oscillating frequency. This frequency shift is used to calculate the mass of butyl acrylate adsorbed using the Sauerbrey equation. See, for example, G. Sauerbrey, Zeitschrift für Phys. 1959, 155, 206, which is incorporated by reference in it entirety. The calculated mass is then converted to a volume using the density of butyl acrylate (0.894 g/mL). A series of adsorption measurements is performed across a range of butyl acrylate p/pₛₐₜ (0.05 to 0.45) and the data is plotted according to a linearization of the BET equation (G. Fagerlund, Matériaux Constr. 1973, 6, 239, which is incorporated by reference in its entirety)

$$\frac{p}{v ( {p}_{sat} - p )} = \frac{c - 1}{{v}_{m} c} ( \frac{p}{{p}_{sat}} ) + \frac{1}{{v}_{m} c} \qquad ( S3 )$$

where p is the pressure of butyl acrylate, pₛₐₜ is the butyl acrylate saturation pressure at the QCM crystal temperature (4.89 Torr), c is the BET constant, and vₘ is the volume of an adsorbed butyl acrylate monomer on a scaffold-coated QCM crystal. A plot of

$$\frac{p}{v ( {p}_{sat} - p )} vs \frac{p}{{p}_{sat}}$$

can be fit to a line (FIG. 9C) and the slope and intercept of this line can be used to calculate vₘ.

$${v}_{m} = \frac{1}{slope + intercept} \qquad ( S4 )$$

Finally, vₘ is converted into a specific surface area using

$${\overline{A}}_{BET} = \frac{{NA}_{m}}{\tilde{V} {m}_{s}} ( {v}_{m} - {v}_{m , b} ) \qquad ( S5 )$$

where N is Avogadro's number, Aₘ is the surface area occupied by a butyl acrylate molecule, \tilde{v} is the molar volume of butyl acrylate (143.4 cm³/mol), and mₛ is the mass of scaffold deposited on the QCM crystal (Table 2). υ_{m,b} is the measured butyl acrylate monolayer volume for a bare QCM crystal (0.15 nL). Aₘ is estimated to be 0.42 nm²/molecule using (S. Lowell, et al., Characterization of Porous Solids and Powders: Surface Area, Pore Size and Density, Springer, Dordrecht, 2004, which is incorporated by reference in its entirety)

$${A}_{m} = 1.091 {( \frac{\tilde{V}}{N} )}^{\frac{2}{3}} \qquad ( S6 )$$

Following the above procedure, Ā_{BET} for fs and GO films employed herein were computed (Table 4).

TABLE 4
HIG Scaffolds BET Surface Area from
Butyl Acrylate Adsorption Experiments

| Scaffold | vₘ (nL) | mₛ (μg) | Ā_{BET} (m²/g) |
|---|---|---|---|
| fs | 1.5 | 13.8 | 170 |
| GO | 0.14 | 5.7 | 0 |

TABLE 5
Comparison of bare fs and fs/NP2 stability*

| | Q_{G} | | n_{G} | |
|---|---|---|---|---|
| | Background | Response | Background | Response |
| Sample | Drift (%) | Decay (%) | Drift (%) | Decay (%) |
| Bare fs | 60.8 | 19.4 | 6.8 | 58.3 |
| fs/NP2 | 6.7 | 4.5 | 1.4 | 19.5 |

*Note:
results are for 14 h test including 5 cycles of 2 ppm GA exposure at 72% RH

TABLE 6
Comparison of GO and GO/NP2 stability.*

| | Q_{B} | | n_{B} | |
|---|---|---|---|---|
| | Background | Response | Background | Response |
| Sample | Drift (%) | Decay (%) | Drift (%) | Decay (%) |
| GO | 6.8 | 16.9 | 7.1 | —** |
| GO/NP2 | 4.5 | 7.3 | 0.1 | 11.8 |

*Results are for 14 h test including 5 cycles of 2 ppm GA exposure at 72% RH
**Indicates no response detectable

Deodorant

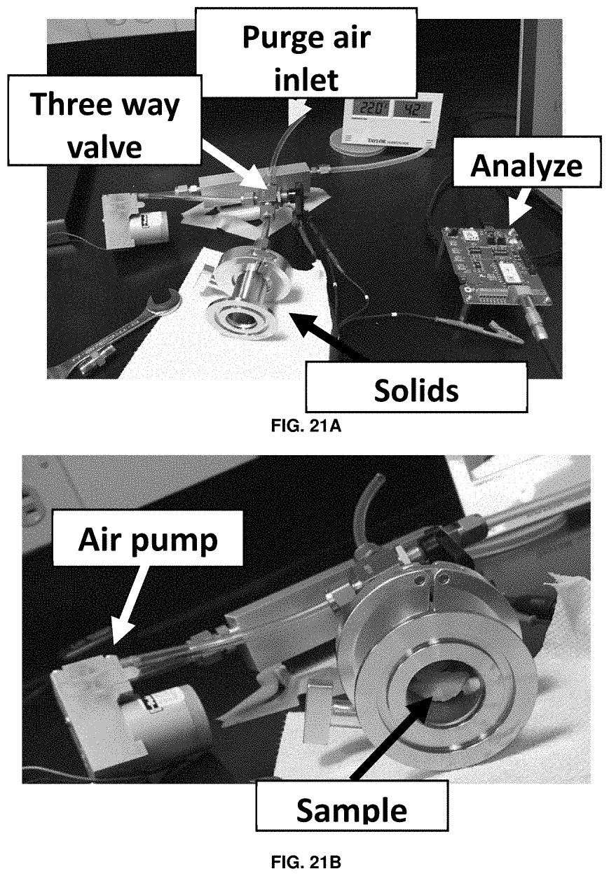

The test setup consisted of the following, as shown in FIGS. 21A-21B. A solids loading container was connected by plastic tubing to a 3-way ball valve. The 3-way ball valve was connected at its other termini to: (1) tubing that leads to ambient air; and (2) a small sized DC air pump. The DC pump inlet was connected to the 3-way ball valve and its outlet connected to a sensors chamber containing HIG sensors. The sensors chamber was connected by air flow to the DC pump at its inlet and connected to ambient at its outlet. The sensors chamber was additionally composed of an aluminum piece that fits tightly over a plastic bus connector. The bus connector contains sensors and was connected by way of a breadboard to a small sized impedance analyzer. The impedance analyzer was connected to and controlled by a laptop computer. For these tests, signal is shown in terms of impedance magnitude (Zmag) and phase angle at 1 kHz. Ambient temperature (˜22° C.) and humidity (˜40% RH) were measured using a commercial desk monitor.

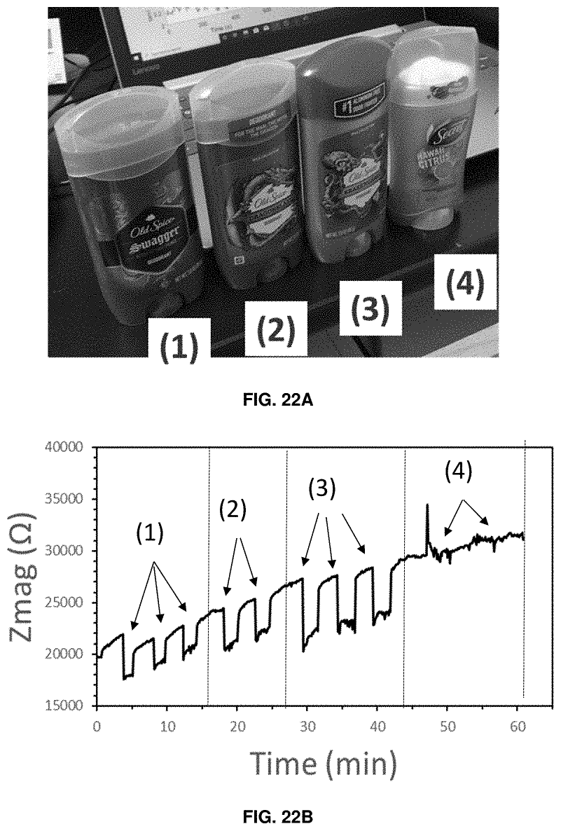

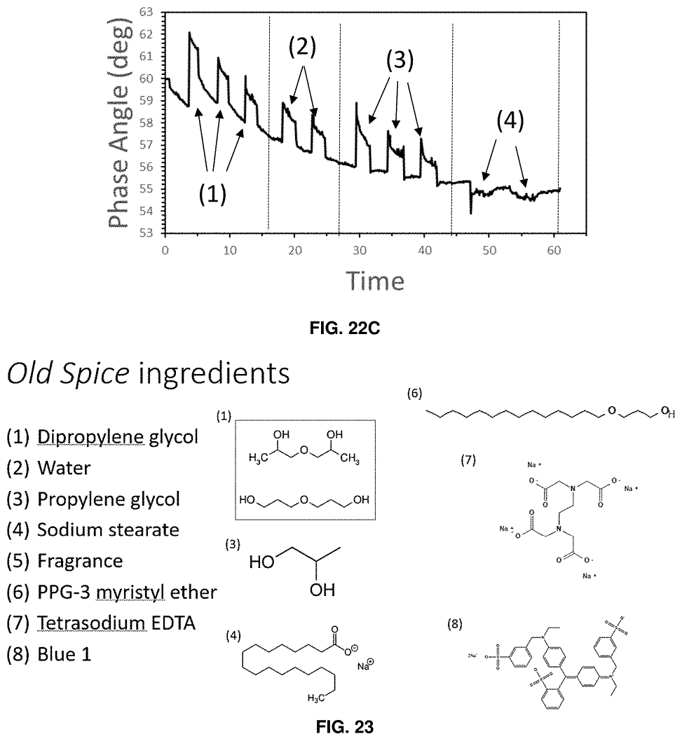

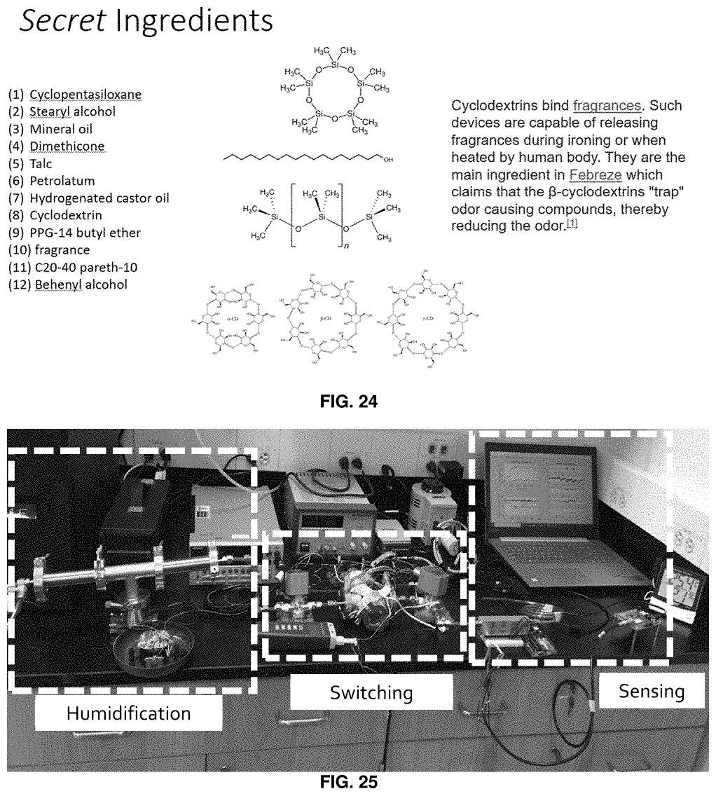

The analytes tested were released at room temperature (no heating) from two different brands of deodorant—Old Spice® and Secret® (FIG. 22A). Approximately the same volume of deodorant was cut from a deodorant stick using a razor blade and placed in the solids loading container to be tested. The released VOCs comprise a mixture that includes various fragrance molecules (FIG. 23 and FIG. 24).

The tests were conducted at ambient temperature (˜22° C.). For these tests, humidity was not enforced. Rather, all tests are accomplished at ambient RH, which was approximately 40% RH.

For these tests, a Type II sensor composed of a graphene oxide (GO) film coated in a nanoscale (<30 nm) film of iCVD polymer polybutyl acrylate, NP1, was used.

The VOCs exposure experiment proceeded as follows. First, a solid sample of deodorant was placed in the solids loading chamber. After this, the 3-way valve was adjusted such that the ambient (purge) air inlet is connected to the DC pump. Next, the DC pump was turned on, and the small analyzer is turned on and programmed to measure sensor impedance spectra at regular time intervals (6 s/cycle). This mode of operation permitted a flow of ambient air and no VOCs to be driven into the sensors chamber. Next, at selected intervals (every 2-3 min), the 3-way valve was adjusted such that the solids loading chamber outlet was connected to the DC pump. This mode of operation permitted a flow of ambient air plus the VOCs released from the solid material to be drawn into the sensors chamber. After waiting for a chosen time interval (˜5 min), the 3-way valve was returned to the ambient air purge position to complete the VOCs exposure experiment.

The key results of this test were as follows (FIGS. 22B and 22C).
This test demonstrated that HIG sensors can sense VOCs released from two brands of deodorant. It is notable that this was done without assistance of sample heating to release more VOCs.
This test demonstrated that detection could be done at more moderate humidity as low as 40%.
This test demonstrated very fast response times (<10 s).
Using just one HIG sensor, two brands of deodorant were clearly distinguished Old Spice® (high response) and Secret® (low response).

Coffee

The test setup for coffee classification was more complex than that employed for deodorant sensing. The test setup can be decomposed into 3 modules, shown in FIG. 25, the humidification module, the switching module and the sensing module.

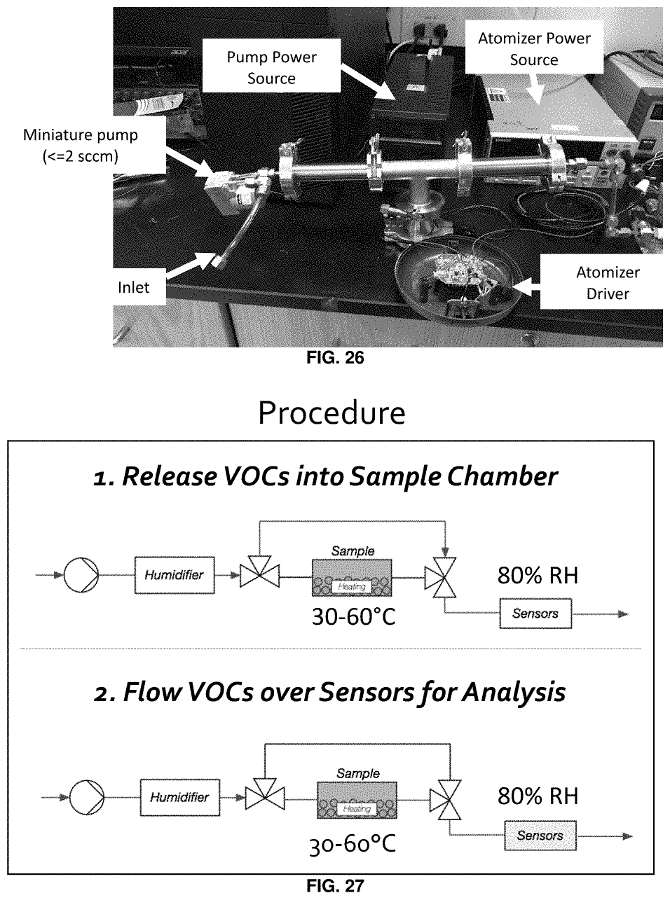

As shown in FIG. 26, the humidification module exists to supply water to ambient air stream before it reaches the sample and sensing chambers. Flow of air to the humidification chamber was supplied by a small DC pump. Humidification was accomplished using a commercially available atomizer and driver. The power supplied to the atomizer was controlled using a programmable power source from 0-22 V, permitting control of extent of flow humidification during an experiment. Flow rate remained constant throughout the experiment.

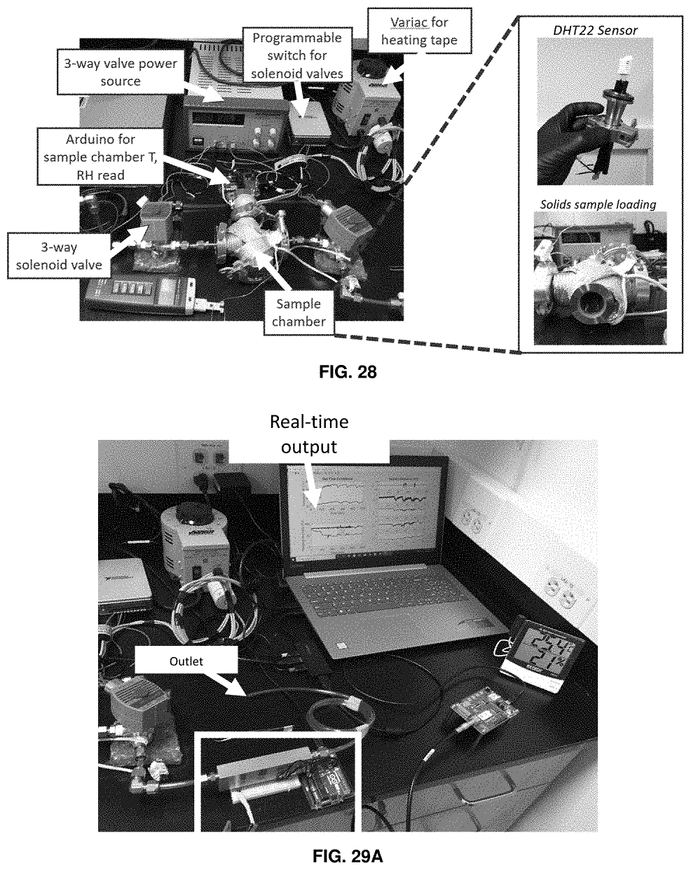

As shown in FIG. 28, the switching module exists to permit two flow conditions (shown in FIG. 27). The first condition is one in which there are no VOCs in flow (direct from humidification module), and the second condition is one in which VOCs in flow that are released from a sample contained within temperature-controlled sample chamber (30-60° C.). Switching of flows was programmatically controlled using a LabView VI that controls a switch that enables two 3-way solenoid valves to be turned on or off. The sample chamber was heated with heating tape, and the surface temperature is monitored with a thermocouple. The internal temperature and humidity of the sample chamber was monitored in-situ using a single sensor placed in the sample chamber.

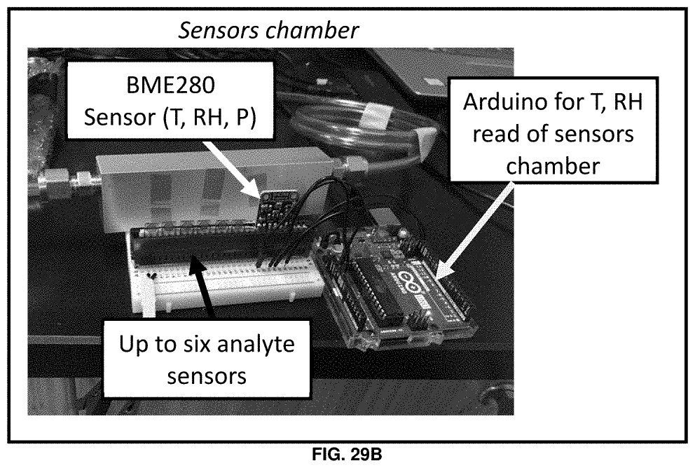

Referring to FIGS. 29A-29B, the sensing module is the terminal module in the test setup and includes a sensing chamber identical to that used in deodorant tests. However, for these tests, a combined temperature and humidity sensor is placed within the chamber along with HIG sensors, and its humidity measurement is used to drive a PID controller that modulates the power supplied to the atomizer driver within the humidification module. VOCs sensing was accomplished with a small analyzer using the same procedure as described for deodorant sensing. Note that for coffee tests, signal is shown in terms and complex dielectric constant at either 1 Hz or 10 Hz.

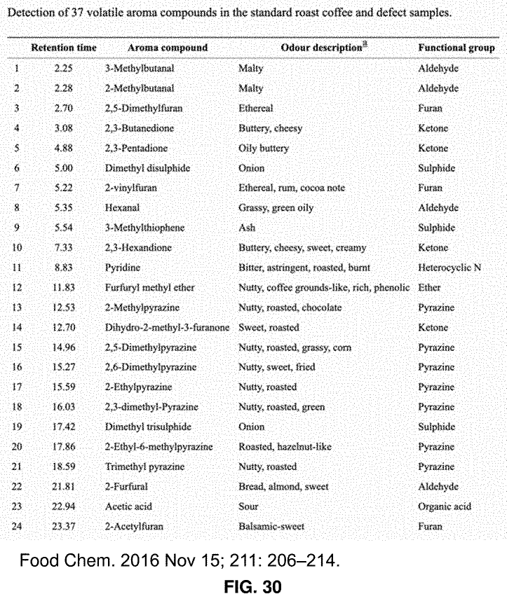

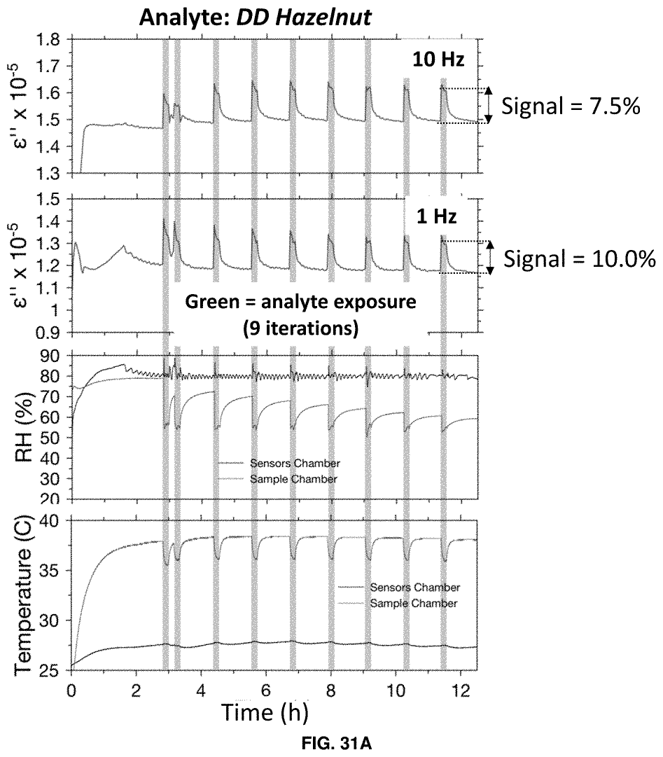

The analytes tested were VOCs released as a rich mixture from 3 different varieties of Dunkin' Donuts® coffee (Caramel, Hazelnut, and Original Blend) when heated. Some potential components are provided in FIG. 30.

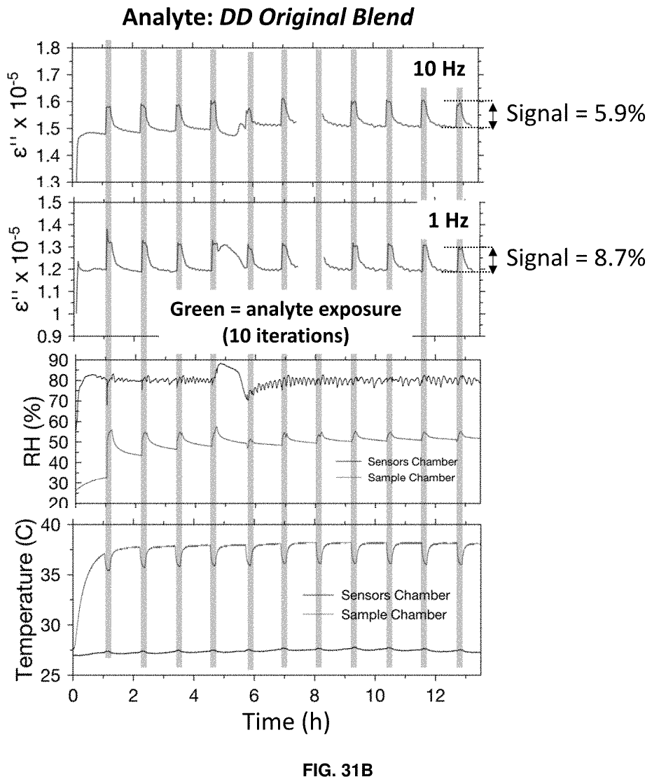

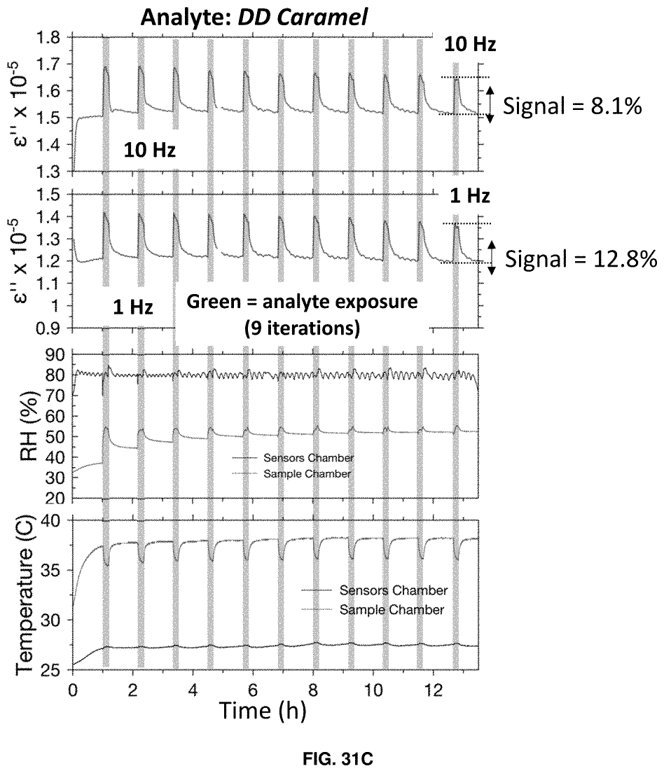

Using the PID control system, the sensors chamber humidity was controlled to a setpoint of 80% relative humidity (RH), as demonstrated in results for a Type II sensor composed of PBA (‘NP1’) (FIGS. 31A-31C). This setpoint was maintained at <5% variation for most of the test duration. Temperature of the sensors chamber was not controlled, but remained at approximately 27° C. with <1 C variation throughout tests.

For these tests, three Type II sensors were employed differentiated by the iCVD polymer used—PBA (‘NP1’), PCHMA (‘NP2’), PHEMA (‘H100’). The thickness of each iCVD polymer film was <30 nm.

The VOCs exposure experiment proceeds as follows. First, flow at a PID setpoint of 80% RH passes over sensors for at least one hour to permit stabilization of RH to 80%. During this time, the sample chamber was sealed and heated to 38° C. Next, the humid flow was diverted (via solenoid valve switch LabView VI) to pass through the sample chamber and then to the sensor chamber. During this time, sensors responded to VOCs exposure. After 15 min, flow through the sample chamber is ceased once again, and humidified air with no VOCs passed over sensors. VOCs exposure cycles were 1 h 15 min in total duration and consist of 15 min of flow across heated sample containing VOCs followed by 1 h purge with humid flow and no VOCs. The total duration of tests was >12 h and was completely automated.

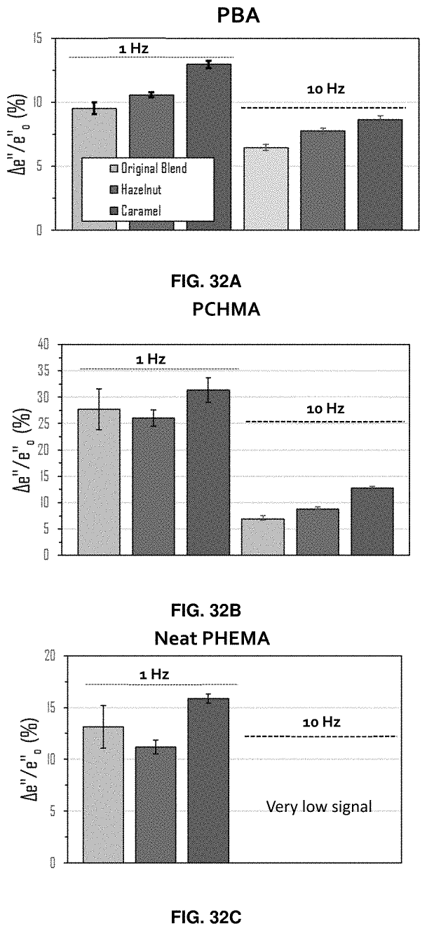

The key results of these tests (FIGS. 32A-32C) were as follows.
An automated system for testing VOCs released from heated solid samples at controlled RH was demonstrated.
Good control of RH was shown throughout the tests.
The presence of response with tight RH control allow for quantification of the response of sensors due only to VOCs introduced to the sensors chamber, and to observe rapid response time (<2 min).
Different sensors demonstrate differences in response (FIGS. 32A-32C). For instance, at 1 Hz, PBA generated the highest response to Caramel followed by Hazelnut and Original Blend, while PHEMA generated highest response for Caramel followed by Original Blend and then Hazelnut.
Both a PCHMA and a PBA sensor can classify the three coffee varieties, as evidenced by non-overlapping error bars (95% confidence intervals) at one or more frequencies tested.

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

What is claimed is:

1. A sensor for detecting an analyte comprising:
a first electrode;
a second electrode; and
a sensor element including a rough-surfaced material having a coating on the surface of the rough-surfaced material, the coated surface having a hydration surface.

2. The sensor of claim 1, wherein the hydration surface includes a surface upon which a thin layer of water forms.

3. The sensor of claim 1, wherein the rough-surfaced material includes inorganic particles.

4. The sensor of claim 3, wherein the inorganic particles include silica.

5. The sensor of claim 1, wherein the hydration surface includes a plurality of capillaries.

6. The sensor of claim 1, wherein the rough-surfaced material includes a capillary-forming material.

7. The sensor of claim 6, wherein the capillary-forming material includes a sheet-forming material.

8. The sensor of claim 1, wherein the water layer sorbs the analyte.

9. The sensor of claim 1 wherein the coating sorbs the analyte.

10. A method of sensing an analyte comprising:
exposing a sensor to an atmosphere having a relative humidity of at least 30%, the sensor including a first electrode, a second electrode, and a sensor element including a rough-surfaced material having a coating on the surface of the rough-surfaced material, the coated surface having a hydration surface;
measuring an electrical property of the sensor to detect the analyte in the atmosphere.

11. The method of claim 10, wherein the atmosphere has a relative humidity of at least 40%, at least 50%, at least 60%, or at least 70%.

12. The method of claim 10, wherein measuring the electrical property of the sensor to detect the analyte in the atmosphere includes measuring the impedance of a water layer on the hydration surface.

13. The method of claim 12, wherein the impedance of the water layer on the hydration surface changes when the water layer sorbs the analyte.

14. The method of claim 10, wherein measuring the electrical property of the sensor to detect the analyte in the atmosphere includes measuring the impedance of a water layer within the hydration surface.

15. The method of claim 14, wherein the impedance of the water layer on the hydration surface changes when the coating sorbs the analyte.

16. The method of claim 10, wherein the analyte is a volatile organic compound.

17. A method of detecting a volatile organic compound comprising:
exposing a sensor to an atmosphere having a relative humidity of at least 30%, the sensor including a first electrode, a second electrode, and a sensor element including a rough-surfaced material having a coating on the surface of the rough-surfaced material, the coated surface having a hydration surface;
measuring an electrical property of the sensor to detect the analyte in the atmosphere includes measuring the impedence of a water layer on or within the hydration surface.

18. The method of claim 17, wherein the atmosphere has a relative humidity of at least 40%, at least 50%, at least 60%, or at least 70%.

19. The method of claim 17, wherein the impedence of the water layer on the hydration surface changes when the water layer sorbs the analyte.

20. The method of claim 17, wherein the impedence of the water layer on the hydration surface changes when the coating sorbs the analyte.

21. The method of claim 17, wherein the volatile organic compound is indicative of a citrus disease.
